(19) United States
(12) Patent Application Publication
(10) Pub. No.: US 2026/0191583 A1
(43) Pub. Date: Jul. 9, 2026

(54) APPARATUS AND METHOD FOR SEPTAL PENETRATION

(71) Applicant: Protaryx Medical Inc., Baltimore, MD (US)

(72) Inventors: James S. GAMMIE, Stevenson, MD (US); David W. MESTER, Venice, FL (US); Timothy P. MACNEIL, Nashville, TN (US); Cameron PURCELL, Santa Rosa, CA (US)

(21) Appl. No.: 19/551,197

(22) Filed: Feb. 26, 2026

Related U.S. Application Data
(63) Continuation-in-part of application No. PCT/US2024/045183, filed on Sep. 4, 2024.
(60) Provisional application No. 63/580,509, filed on Sep. 5, 2023.

(51) Int. Cl. A61B 18/14; A61B 17/00; A61B 18/00; A61M 25/09

(52) U.S. Cl. CPC A61B 18/1492; A61B 2017/00247; A61B 2017/00867; A61B 2018/00279; A61B 2018/00357; A61B 2018/144; A61M 2025/09183

(57) ABSTRACT

In some embodiments, an apparatus includes a sheath defining a lumen and a catheter slidably disposable within the lumen of the sheath and configured to extend distally relative to the sheath. The apparatus includes a dilator slidably disposable within a lumen of the catheter and configured to extend distally relative to the catheter and the sheath. A distal end portion of the sheath has an inner diameter that is less than a maximum outer diameter of the dilator when a portion of the dilator having the maximum outer diameter is disposed proximal to or distal to the distal end portion of the sheath. The apparatus further includes a radiofrequency (RF) guidewire slidably disposable within a lumen of the dilator and configured to extend distally relative to the dilator, the RF guidewire configured deliver energy to a target perforation site of a patient.

### CROSS-REFERENCING TO RELATED APPLICATIONS

This application is a continuation-in-part of PCT Application No. PCT/US2024/045183, filed Sep. 4, 2024, titled “APPARATUS AND METHOD FOR SEPTAL PENETRATION,” which claims priority to and the benefit of U.S. Provisional Patent Application No. 63/580,509, filed Sep. 5, 2023, and titled “APPARATUS AND METHOD FOR SEPTAL PENETRATION,” the disclosure of which is incorporated herein by reference.

### TECHNICAL FIELD

Embodiments are described herein that relate to devices and methods for use in accessing the left side of the heart.

### BACKGROUND

Many diseases and disorders, such as, for example, heart failure, atrial fibrillation, mitral valve disease, and others, specifically impact or are addressable in the left side of the heart. Accordingly, many interventional percutaneous cardiac procedures require access to the left side of the heart, including, for example, electrophysiological procedures, left atrial appendage occlusion procedures, mitral valve repair and replacement procedures, atrial shunt procedures, and many more. In addition to therapeutic interventional procedures, indications for access to the left side of the heart also include diagnostic procedures, including, for example, hemodynamic measurements (e.g., left atrial pressure, trans-mitral pressure gradient, etc.). Minimally-invasive access to the left side of the heart is challenging and not without significant risk.

Some catheter-based procedures access the left side of the heart by puncturing the atrial septum (“AS”) of the heart, which separates the left atrium (“LA”) of the heart from the right atrium (“RA”) of the heart. Such procedures use a catheter containing a sheathed needle, which is advanced from the femoral vein in the groin of the patient to the superior vena cava (“SVC”) through the RA of the heart. The sheathed needle is often a long, stiff-wire needle that has a bend of approximately twenty degrees near its tip. With the catheter assembly disposed within the SVC, the catheter assembly is then slowly withdrawn inferiorly from the SVC and into the RA until its tip rests within the fossa ovalis (“fossa”, “FO”, or “F”). The FO is a thumbprint-sized depression in the wall of the RA and is the thinnest portion of the interatrial septum (i.e., the wall between the RA and LA). Once the operator visualizes contact between the tip of the catheter assembly and the FO, the needle is advanced such that it punctures the FO. With the needle extending from the LA into the RA, a guidewire is advanced through the catheter and into the RA. The needle is then removed from the LA, and a device (e.g., an AFib ablation device, a catheter, percutaneous mitral valve repair delivery system or catheter, as examples) can be inserted into the LA.

Alternative procedures include the use of a blunt needle, electrified by radiofrequency, to puncture or perforate the atrial septum.

The above procedure has significant limitations. It is difficult to learn, time intensive, and prone to premature, misaligned, and inadvertent puncturing of the FO. Further, precisely and accurately locating the FO with the tip of the device is difficult, and if the catheter assembly is withdrawn from the SVC too far, time-intensive procedural steps must be repeated because such a device cannot be moved cephalad or side-to-side without the potential for “whipping” off the FO. Moreover, the shape of the needle may need to be customized or adjusted based on a patient's particular anatomy, thereby further complicating the process.

Furthermore, such catheters are typically very flexible and not very stable within the SVC, and thus easily inadvertently maneuvered out of an ideal position, particularly during normal dynamic cardiac activity. Even more, the needle is not fixed to the catheter, thereby resulting in accidental needle exposure, and possibly inadvertent cardiac puncture (i.e., cardiac perforation), which can be lethal. Further complicating this procedure is potentially distorted or abnormal anatomy due to, for example, aortic or mitral valve disease, leading to changes in the location of the FO and obfuscation of typical anatomical landmarks. Yet even more, for patients undergoing a repeat procedure, the FO may be thickened or scarred, necessitating application of greater puncturing force and increased risk of unintended damage to nearby anatomy.

It can be crucial for many left-heart procedures that the septal puncture is performed in a specific location within the FO. For delivering a replacement mitral valve, for example, it may be important to puncture an inferior portion of the FO, while for a native valve leaflet clip implant procedure, it may be important to puncture a post/mid portion of the FO. Existing systems do not provide for sufficient accurate and precise targeting of an intended puncture site, such as a particular region within the FO. Failure to puncture the septum in a proper location can result in prolonged, unsuccessful, or canceled procedures.

Existing systems can also require an exchange between a device for delivery of a transseptal guidewire and a device to delivery therapy to the right atrium.

Thus, a need exists for improved devices and methods for faster, more stable, safer, more accurate, more precise access, and exchangeless access to the LA.

### SUMMARY

In some embodiments, an apparatus comprises a sheath defining a lumen; a catheter slidably disposable within the lumen of the sheath and configured to extend distally relative to the sheath, the catheter defining a lumen; a dilator slidably disposable within the lumen of the catheter and configured to extend distally relative to the catheter and the sheath, the dilator defining a lumen, a distal end portion of the sheath has an inner diameter that is less than a maximum outer diameter of the dilator when a portion of the dilator having the maximum outer diameter is disposed proximal to or distal to the distal end portion of the sheath; a radiofrequency (RF) guidewire slidably disposable within the lumen of the dilator and configured to extend distally relative to the dilator, a proximal end portion of the RF guidewire configured to be coupled to an RF generator to deliver RF energy to a distal end portion of the RF guidewire and to a target perforation site of a patient.

In some embodiments, an apparatus comprises a sheath defining a lumen; a catheter slidably disposable within the lumen of the sheath and configured to extend distally relative to the sheath, the catheter defining a lumen and having an end effector at a distal end thereof, the end effector configured to transition from first configuration in which the end effector has a first diameter to a second, atraumatic configuration in which the end effector has a second diameter greater than the first diameter; a dilator slidably disposable within the lumen of the catheter and configured to extend distally relative to the catheter and the sheath, the dilator defining a lumen; a radiofrequency (RF) guidewire slidably disposable within the lumen of the dilator and configured to extend distally relative to the dilator, the distal end portion of the RF guidewire configured to be activated to deliver RF to perforate a target perforation site of a patient.

In some embodiments, an apparatus comprises a sheath; a dilator slidably disposable within the sheath and configured to extend distally relative to the sheath, the dilator defining a lumen, a distal end portion of the sheath having a lower durometer than a portion of the sheath proximal to the distal end portion of the sheath such that an inner diameter of the distal portion of the sheath is configured to expand and contract laterally according to an outer diameter of the dilator; and a radiofrequency (RF) guidewire slidably disposable within the lumen of the dilator and configured to extend distally relative to the dilator, the distal end portion of the RF guidewire configured to be activated to deliver RF to perforate a target perforation site of a patient.

In some embodiments, a method comprises navigating a catheter assembly to a right atrium of a patient, the catheter assembly including a sheath and a catheter slidably disposed in the sheath, the catheter including an end effector on a distal end thereof; disposing the end effector of the catheter distal to a distal end of the sheath such that the end effector transitions from a first configuration in which the end effector has a first diameter to a second, atraumatic configuration in which the end effector has a second diameter greater than the first diameter; tenting a target perforation site of a septum with the end effector; and perforating, with the target perforation site tented, the target perforation site with the guidewire.

### BRIEF DESCRIPTION OF THE DRAWINGS

FIG. 1A is a schematic illustration of a septum puncture device, disposed in a delivery configuration, according to an embodiment.

FIG. 1B is a schematic illustration of the septum puncture device of FIG. 1A, disposed in a deployed configuration.

FIG. 2A is a schematic illustration of the septum puncture device of FIG. 1A, disposed in the delivery configuration within a right atrium (“RA”) of a heart of a patient, and coupled to a first guidewire extending from an inferior vena cava (“IVC”) of the heart across the RA and into a superior vena cava (“SVC”) of the heart.

FIG. 2B is a schematic illustration of the septum puncture device of FIG. 1A, disposed in the deployed configuration and such that it has accessed and delivered to the LA a second guidewire.

FIG. 3 is a flowchart illustrating a method of using a septum puncture device to access a left atrium of a heart of a patient, according to an embodiment.

FIGS. 4A-5H illustrate a septum puncture device, and deployment thereof, with slight variations, according to an embodiment.

FIGS. 6A-6D schematically illustrate a septum puncture device, and deployment thereof, according to an embodiment.

FIGS. 7A-7F illustrate a septum puncture device, and deployment thereof, according to an embodiment.

FIG. 8A is a side view of the side or inner guide catheter of the septum puncture device of FIGS. 7A-7F, FIG. 8B is an end view of the side or inner guide catheter, FIG. 8C is a longitudinal cross section through a distal portion of the side or inner guide catheter, and FIG. 8D is a longitudinal cross-section of the side or inner guide catheter along section 8B-8B of FIG. 8A.

FIGS. 9A-9E are schematic illustrations of the deployment of a septum puncture device accordingly to an embodiment.

FIGS. 10A-10C are schematic block diagrams of a septum puncture device in a navigating configuration, a tenting configuration, and a dilating configuration, respectively, according to embodiments.

FIG. 11A is a schematic block diagram of a distal end of a septum puncture device including a sheath, a catheter, and a dilator, according to embodiments.

FIG. 11B is a front cross-sectional view showing the catheter disposed around the dilator, and the sheath disposed around the catheter, according to embodiments.

FIG. 12A is an illustration of a distal end of a septum puncture device including a catheter, a dilator, and a guidewire all in a deployed configuration, according to embodiments.

FIG. 12B is a side cross-sectional view of the distal end of the septum puncture device in the deployed configuration.

FIG. 12C is a side cross-sectional view of the distal end of the septum puncture device showing the dilator extended from a distal end of the catheter and the sheath, according to embodiments.

FIG. 13A image of a septum puncture device showing a position of radiopaque markers, according to embodiments.

FIG. 13B shows the radiopaque markers of the septum puncture device when imaged with fluoroscopic imaging, according to embodiments.

FIGS. 14A-14B show a guidewire for navigating a septum puncture device the guidewire including a pigtailed distal end, according to embodiments.

FIG. 15 shows markers of the septum puncture device when imaged with fluoroscopic imaging, according to embodiments.

FIG. 16A shows an end effector of a catheter of a septum puncture device, according to embodiments.

FIG. 16B shows the end effector of the catheter as visible during ultrasound imaging, according to embodiments.

FIG. 16C shows a diagram of the catheter and the end effector, according to embodiments.

FIG. 17 shows a diagram of a sheath of a septum puncture device configured to have a catheter, dilator, and guidewire extended therethrough, according to embodiments.

FIG. 18 shows a distal end of the septum puncture device, according to embodiments.

FIG. 19 shows a catheter of a septum puncture device in a sheathed position and a deployed position, according to embodiments.

FIGS. 20A-20G show different configurations of the septum puncture device of FIG. 19 during a procedure of puncturing a septum of a heart, according to embodiments.

FIGS. 21A-21K show a method for puncturing a septum of a heart using the septum puncture device of FIGS. 12A-12C, according to embodiments.

FIG. 22A shows a cross-sectional side view of a septum puncture device including an outer dilator disposed inside a sheath and around a catheter, according to embodiments.

FIG. 22B shows a cross-sectional front view of the septum puncture device of FIG. 22A, according to embodiments.

FIG. 23 is a flow chart diagram of an example method of puncture a septum of a heart of a patient using a septum puncture device, according to embodiments.

FIGS. 24A-24B is a schematic block diagram of a distal portion of a septum puncture device, according to embodiments.

FIG. 25 is an image depicting a distal portion of a septum puncture device, according to embodiments.

FIG. 26 is a graph comparing change in radius of sheaths of septum puncture devices over a distance from a distal tip of the sheaths, according to embodiments.

FIGS. 27A-27B show a catheter unconstrained and constrained, respectively, by a loading tool, according to embodiments.

FIGS. 28A-28C show loading of the catheter of FIGS. 27A-27B into a proximal end of a sheath of a puncture septum device, according to embodiments.

FIG. 29 is a schematic block diagram of a method of loading a catheter into a sheath, according to some embodiments.

### DETAILED DESCRIPTION

Devices and methods are described herein for use in accessing the left side of the heart (e.g., LA) from the right side of the heart (e.g., RA) without requiring open-heart surgery. The methods described herein are minimally invasive and utilize a septum puncture device to access the left side of the heart in a safe (e.g., atraumatic), efficient, timely, accurately and precisely located and repeatable manner.

As used herein, the terms “proximal” and “distal” refer to the direction closer to and away from, respectively, an operator (e.g., a surgeon, physician, nurse, technician, etc.) who would insert the septum puncture device into the patient, with the tip-end (i.e., distal end) of the device inserted inside a patient's body first. Thus, for example, the end of a main shaft described herein first inserted inside the patient's body would be the distal end, while the opposite end of the main shaft (e.g., the end of the main shaft being manipulated by the operator) would be the proximal end of the main shaft.

As used herein, the terms “advance,” “advanced,” and “advancing” each refer to distal movement. Advancing a device within a patient's vasculature, for example, refers to moving at least a portion of the device distally within the patient's vasculature. Similarly, as used herein, the terms “withdraw,” “withdrawn,”, and withdrawing” each refer to proximal movement. Withdrawing a device within a patient's vasculature, for example, refers to moving at least a portion of the device proximally within the patient's vasculature. In some instances, advancing and withdrawing can refer to relative movement of the device itself. Advancing a side catheter, for example, can refer to moving a side catheter distally relative to a side catheter guide to which the side catheter is movably coupled. Similarly, withdrawing the side catheter, for example, can refer to moving the side catheter proximally relative to the side catheter guide to which the side catheter is movably coupled.

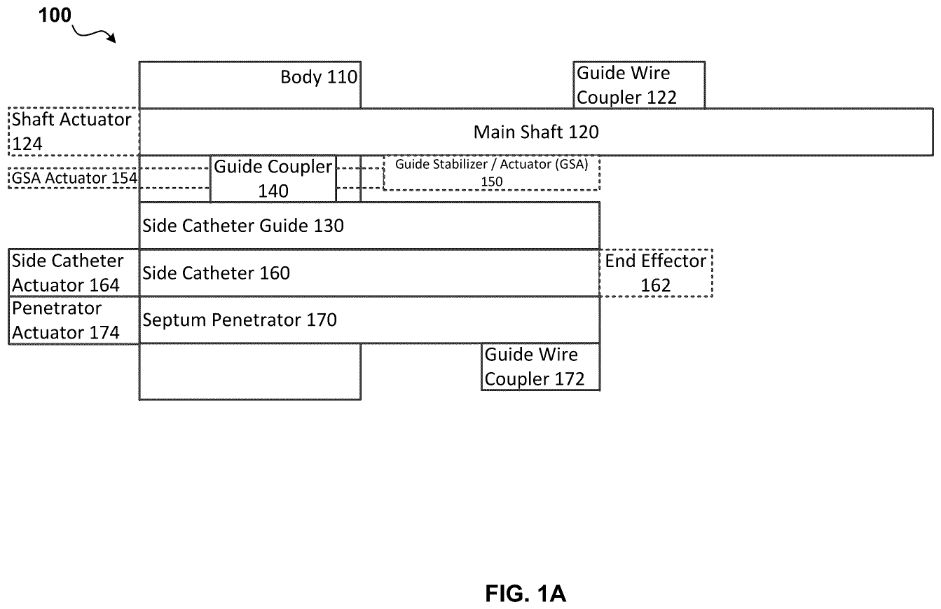

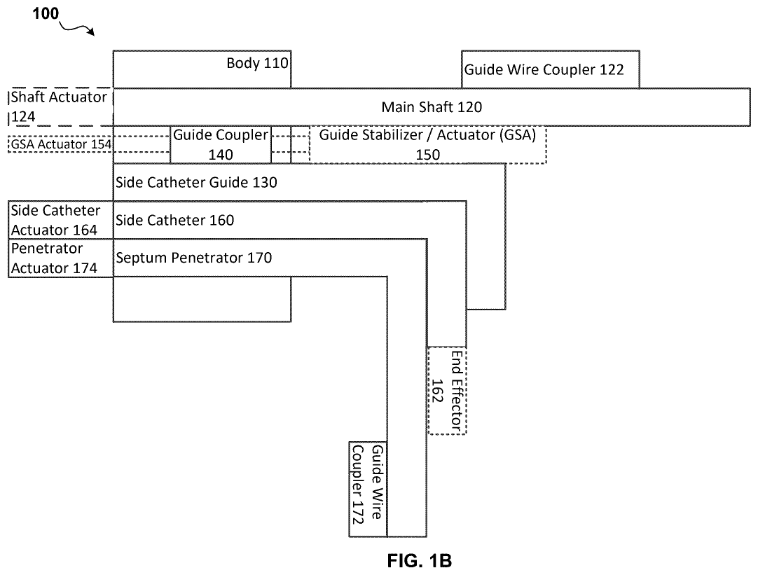

A septum puncture device 100 can be used to access a left side of the heart (e.g., left atrium) from the right side of the heart (e.g., right atrium) and to deliver a guidewire to the left side of the heart. This device is shown and described schematically, and many specific implementations and alternative embodiments are shown and described, in International Patent Application Publication No. WO 2021/195243, the entire disclosure of which is incorporated by reference herein. As shown in FIG. 1A, the septum puncture device 100 includes a body 110 coupled to a main shaft 120, a side catheter guide 130, a side catheter 160, and a septum penetrator 170. The main shaft 120 is coupled to the side catheter guide 130 via a guide coupler 140, the side catheter guide 130 is coupled to the side catheter 160, and the side catheter 160 is coupled to the septum penetrator 170, as shown in FIG. 1A. The side catheter guide 130 is configured to define a pathway through or across which the side catheter 160 can travel (e.g., be advanced and/or withdrawn). Said another way, and as described in further detail herein, the side catheter guide 130 can be manipulated (e.g., actuated from a delivery state to a deployed state) to guide the side catheter 160 in a desired direction (the actuated or deployed state of the side catheter guide 130 is shown in FIG. 1B), e.g., towards the left atrium.

As described in further detail herein, the guide coupler 140 can couple the side catheter guide 130 to the main shaft 120 to minimize or prevent relative translational movement between the main shaft 120 and the side catheter guide 130, but to allow relative rotational movement between the main shaft 120 and the side catheter guide 130, as illustrated schematically in FIG. 1B. In this manner, the guide coupler 140 can facilitate transition of the side catheter guide 130 from a delivery configuration (e.g., parallel to or substantially parallel to the main shaft 120), e.g., for insertion through the patient's vasculature and into the RA, to a deployed configuration such that a distal end of the side catheter guide 130 is deflected laterally (e.g., perpendicular or substantially perpendicular) relative to the main shaft 120, e.g., towards the patient's left atrium (e.g., the FO of the atrial septum). In some embodiments, the guide coupler 140 can be a hinge to facilitate lateral deflection of the side catheter guide 130 relative to the main shaft 120, as described in further detail herein. In such embodiments, for example, a distal force can be applied to a proximal end portion of the side catheter guide 130, thereby causing the hinge to rotate and cause a distal end portion of the side catheter guide (i.e., a portion of the side catheter guide 130 that extends distal to the guide coupler 140) to laterally deflect. In some implementations, the amount of lateral deflection or the defined between the side catheter guide 130 and the main shaft 120 after such lateral deflection is adjustable by the operator intra-procedure, i.e., in real-time, such that, for example, the operator has procedural flexibility when locating the target puncture location.

In some implementations, one or more of the main shaft 120, the side catheter guide 130, or the side catheter 160 can have a circular cross-sectional shape, while in other implementations, one or more of the main shaft 120, the side catheter guide 130, or the side catheter 160 can have a non-circular cross-sectional shape. In some instances, for example, the main shaft 120 and the side catheter guide 130 can have circular cross-sectional shapes, and can be operably coupled together, as discussed in further detail herein, such that the main shaft 120 and the side catheter guide 130 are at least partially disposed side-by-side (e.g., during delivery). In other instances, for example, the main shaft 120 may have a non-circular cross-section (e.g., a half-moon shape, c-shape a convex or concave shape, or any other suitable noncircular cross-sectional shape) such that when coupled to the side catheter guide 130, a portion of the side catheter guide 130 can be nestled within a space defined at least in part by the non-circular curvature of the main shaft 120. In this manner, the collective cross-sectional area, footprint, diameter, etc. of the main shaft 120 and side catheter guide 130 can be reduced. In some instances, a similar relationship can be had by the main shaft 120 and the side catheter 160 (e.g., in embodiments in which a septum puncture device does not have a side catheter guide).

In some embodiments, the septum puncture device 100 includes a side catheter guide stabilizer/actuator (“GSA”) 150 (also referred to herein as “guide stabilizer/actuator”), and a GSA actuator 154 operably coupled to the GSA 150 and configured to actuate the GSA 150. In some implementations, the GSA 150 can be configured to stabilize (e.g., laterally, axially (proximally or distally), e.g., with respect to the main shaft 120) the side catheter guide 130 to facilitate the side catheter's 160 engagement with the FO and the septum penetrator's 170 penetration of the FO. In this manner, the guide coupler 140 can laterally deflect the side catheter guide 130, and the GSA 150 can stabilize the side catheter guide 130 (and in turn the side catheter 160, optional end effector 162, and septum penetrator 170) to optimize subsequent penetration of the septum and access to the left atrium. In some implementations, in addition to or instead of stabilizing the side catheter guide 130, the GSA 150 can be configured to laterally deflect (e.g., laterally deflect in addition to the lateral deflection caused or facilitated by the guide coupler 140, as described above) the side catheter guide 130 (and in turn the side catheter 160 and septum penetrator 170, given their coupling to the side catheter guide 130). In this manner, in some implementations, the guide coupler 140 and the GSA 150 can collectively laterally deflect and stabilize the side catheter guide 130 (and in turn the side catheter 160, optional end effector 162, and septum penetrator 170) to optimize subsequent penetration of the septum and access to the left atrium.

The GSA 150 can be manipulatable in any manner suitable to provide the above-described functionality. In some embodiments, for example, the GSA 150 can be a balloon, and as such, it can be configured to be inflatable and deflatable. In such embodiments, the GSA 150 can be fluidically coupled to a lumen extending from the GSA 150 to the GA actuator 154 such that the GA actuator 154 can selectively deliver fluid to the GA actuator 154 to inflate the GSA 150 (i.e., deploy the GSA 150), and selectively withdraw fluid from the GSA 150 to deflate the GSA 150 for removal of the GSA 150 from the heart (e.g., after left atrium access has been achieved).

In embodiments in which the GSA 150 is a balloon, the balloon can have any shape and size suitable to perform the desired functions described herein. In some embodiments, for example, the balloon can be cone-shaped, while in other embodiments, it can be at least partially concave, convex, circular, oval, or the like. Further, in some embodiments, the balloon can have one or more lobes, e.g., it can be bi-lobed or tri-lobed, to, for example, allow blood flow along the balloon and past the device. Further, the balloon can have additional features configured to improve stabilization of the side catheter guide 130 (e.g., improve coupling between the balloon and the side catheter guide 130). In some embodiments, for example, a balloon can have dimples, protrusions, ridges, adhesives, etc.

The balloon can be formed of any material or combination of materials suitable to perform its functionality described herein. In some embodiments, for example, the balloon can be formed of one or more of Polyethylene, Polyethylene terephthalate (“PET”), a polymer, a thermoplastic polymer, an elastomer, nylon, polyurethane, any non-compliant material, etc. The balloon can be configured to be inflated to any suitable pressure, e.g., from about 2 ATM to about 20 ATM, as an example. In some instances, higher inflation pressures can result in greater or improved rigidity of the balloon, thereby providing better stabilization of the side catheter guide, side catheter, septum penetrator, etc.

The GSA 150 can be formed of any material suitable to perform its functions described herein. In some embodiments the GSA 150 can include or be formed of shape memory material (e.g., Nitinol) and configured to be transitioned between a delivery/withdrawal configuration in which the GSA 150 is constrained, compressed, or otherwise placed in a relatively small arrangement, and a deployed configuration in which the GSA 150 is unconstrained, expanded, or otherwise placed in a larger arrangement sufficient to laterally deflect or stabilize the side catheter guide 130 as described in further detail herein.

Similar to the guide coupler 140, in some embodiments, the GSA 150 can include or be formed of radiopaque material to assist the operator in locating that portion of the septum puncture device 100 before, during, or after deployment. In this manner, the operator can in real time selectively position the septum penetrator 170 in a position suitable to penetrate the FO upon actuation of the septum penetrator 170. In embodiments in which the GSA 150 is a balloon, for example, in some instances the GSA 150 can be inflated with a contrast agent (or a combination of a contrast agent and another fluid, such as saline) to provide visualization (e.g., under any suitable imaging modality) for the operator when the GSA 150 is disposed within the patient.

As described in further detail herein, with the side catheter guide 130 laterally deflected and stabilized at a suitable angle relative to the FO or the main shaft 120, and with (1) one or more landmark portions of the septum puncture device 100 and (2) a desired puncture location (e.g., the FO) on the septum visible to the operator from outside the patient, the operator can manipulate the main shaft 120 translationally or rotationally in any suitable manner to align the side catheter guide 130 with the FO.

Further as shown in FIG. 1A, the septum puncture device 100 includes a guidewire coupler 122 configured to couple the main shaft 120 to a guidewire (not shown in FIG. 1A) to facilitate delivery of the septum puncture device 100 into a patient (e.g., through the vasculature of the patient) and to the patient's heart, and a guidewire coupler 172 configured to couple a guidewire (not shown in FIG. 1A) to the septum penetrator 170, to facilitate delivery of that guidewire to the left side of the heart (e.g., the left atrium).

Further as shown in FIG. 1A, the septum puncture device 100 optionally includes a shaft actuator 124 operably coupled to the main shaft 120 and configured to actuate the main shaft 120 to advance or withdraw the main shaft 120 relative to the body 110. The septum puncture device 100 further includes (1) a side catheter actuator 164 operably coupled to and configured to actuate the side catheter 160 to advance or withdraw the side catheter 160, thereby transitioning the side catheter 160 between a delivery configuration and a deployed configuration (the side catheter 160 shown in an actuated or deployed configuration in FIG. 1B), and a (2) a septum penetrator actuator (or “penetrator actuator”) 174 to actuate the septum penetrator 170 to advance or withdraw the septum penetrator 170, thereby transitioning the septum penetrator 170 between a delivery configuration and a deployed configuration (the septum penetrator 170 shown in an actuated or deployed configuration in FIG. 1B), as described in further detail herein.

Further as shown in FIG. 1A, the septum puncture device 100 optionally includes a GSA (“GA”) 150 coupled to the main shaft 120. The optional GSA 150 is operably coupled to a GA actuator 154 that is configured to actuate the GSA 150, as described in further detail herein.

Further as shown in FIG. 1A, the septum puncture device 100 optionally includes an end effector 162 coupled to and extending distally from the side catheter 160. The end effector 162 is configured to facilitate subsequent puncture through a target puncture location, such as, for example, the FO of the septum of the heart. The end effector 162 can be configured, for example, to contact or tent the FO, as described in further detail herein. Such contact or tenting of the FO can, for example, reduce or minimize the force required to penetrate the FO and/or provide for improved force distribution to the FO. The end effector 162 can be configured to prevent inadvertent puncturing of and/or damage to the FO with the end effector 162.

In some embodiments, the end effector 162 is formed of or includes a radiopaque material such that the end effector 162 can be visualized when within the heart from outside the patient under any suitable imaging modality (e.g., fluoroscopy, echocardiography, etc.), to facilitate an operator in deploying the end effector 162, e.g., locating the end effector 162 within the heart or relative to the FO in preparation for deploying the septum penetrator 170.

In some embodiments, the end effector 162 can include multiple configurations, e.g., a delivery or withdrawal configuration, in which the end effector 162 is configured to be routed through the patient's vasculature, and a deployed configuration in which the end effector 162 is configured to facilitate subsequent penetration of the FO, as described in further detail herein. In such embodiments, for example, the end effector 162 can be delivered to the heart in a compressed, deflated, or otherwise relatively small configuration, and then transitioned into a deployed configuration in which it is expanded, inflated, or otherwise increased in size to then contact or tent the FO. Further, in some embodiments, after deployment of the end effector 162, the end effector 162 can be transitioned to a withdrawal configuration (which can be the same as or similar to its delivery configuration) in which the end effector 162 is in a compressed, deflated, or otherwise small configuration to assist in removal of the end effector 162 from the patient.

The end effector 162 can be formed of any suitable material(s) to facilitate its functionality described herein. In some embodiments, for example, the end effector 162 can be formed of shape memory material(s) (e.g., Nitinol) or a polymer, or a combination thereof (e.g., Nitinol coated with a polymer), such that it can be transitioned between a constrained or compressed arrangement (e.g., delivery or withdrawal configuration) and an unconstrained or expanded arrangement (deployed configuration). In some embodiments, for example, the end effector 152 can be or include a balloon such that it can be delivered to the heart in a deflated arrangement and then inflated (e.g., via an inflation lumen fluidically coupled to and extending proximally from the end effector 162, not shown) to a deployed configuration. Various further embodiments of an end effector are described in further detail below.

Each of the main shaft 120, the guidewire coupler 122, the side catheter guide 130, the guide coupler 140, the optional GSA 150, the side catheter 160, the septum penetrator 170, and the guidewire coupler 172 are translatable (e.g., distally advanceable and/or extendable, and proximally withdrawable and/or retractable) relative to the body 110. The side catheter 160 is translatable relative to the side catheter guide 130, and the septum penetrator 170 is translatable relative to the side catheter 160, as described in further detail herein.

The septum penetrator 170 can be sized, shaped, and formed of any material suitable to effectively penetrate and traverse a target tissue such as the FO. In some embodiments, for example, the septum penetrator 170 can be a needle. In some embodiments, the septum penetrator 170 can be a non-coring needle (e.g., a needle with a sharp tip that has a cutting edge, such as, for example, a Quincke-type needle). In some embodiments, the septum penetrator 170 can have variable material properties. In such embodiments, for example, a distal portion of the septum penetrator 170 can have a stiffness greater than a stiffness of a portion proximal to that distal portion. In this manner, the stiffer distal portion can be configured for penetration through the septum, while the portion proximal can be configured for delivery through the patient's vasculature. In some embodiments, the septum penetrator 170 can be solid-tipped and can be electrified with radiofrequency (“RF”) energy to puncture the FO.

The septum penetrator 170 can have any suitable length, for example, any length suitable to reach the LA. In some embodiments, for example, the septum penetrator 170 can have an effective length (i.e., the length extendable from the distal end of the side catheter 160 (or from the distal end of the end effector 162) of about 5 mm to about 25 mm. In some instances, an effective length of the septum penetrator 170 can be about 8 mm or about 10 mm, or any length therebetween. In some embodiments, the septum penetrator 170 can contain or be configured to receive a stylet to limit or minimize tissue coring. In some embodiments, the septum penetrator 170 can include a pressure transducer (not shown) configured to monitor pressure through a lumen of the septum penetrator 170. In some embodiments, a port or leur lock can be incorporated into the septum puncture device 100 to flush the septum penetrator 170.

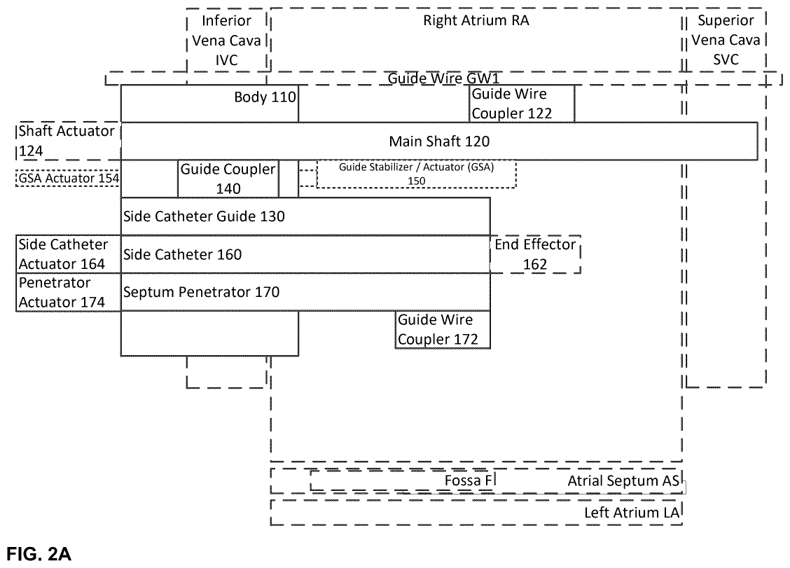

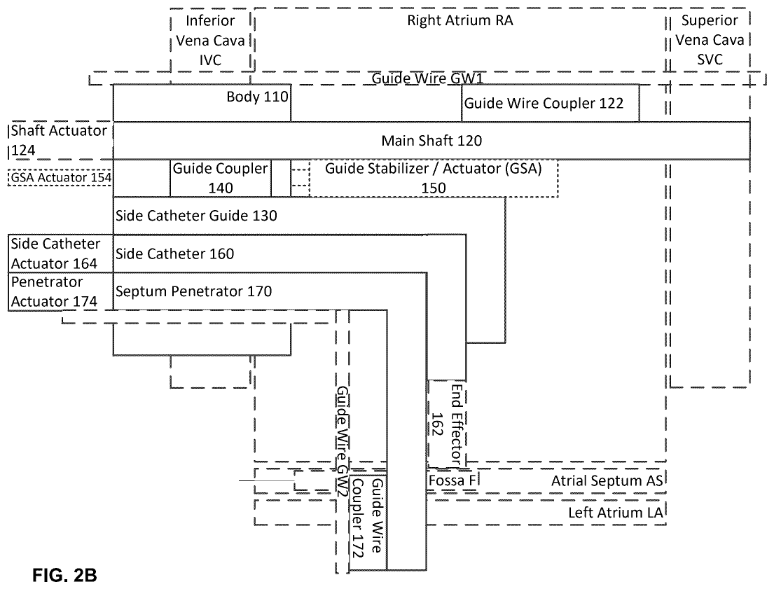

Turning to FIGS. 2A and 2B to describe the septum puncture device 100 (1) in context with the anatomy of a patient and (2) in a sample procedure to access the LA of the patient, FIG. 2A is a schematic illustration of the septum puncture device 100 disposed in a delivery configuration within the RA of the heart and coupled to a first guidewire GW1 extending from the IVC across the RA and into a SVC and FIG. 2B is a schematic illustration of the septum puncture device 100 disposed in a deployed configuration and such that it has accessed and delivered to the LA a second guidewire that can be used to provide subsequent access to the LA.

In use, prior to introducing into the patient the septum puncture device 100, a guidewire GW1 can be inserted through an entry site of the patient (e.g., femoral vein puncture site) (not shown) and advanced through the patient's vasculature across the IVC and RA, and into the SVC using known, suitable techniques for guidewire delivery. With the guidewire GW1 disposed in such a manner, the septum puncture device 100 can be movably coupled to the guidewire GW1 via the guidewire coupler 122 and advanced from the entry site of the patient towards the heart. In some embodiments, the guidewire coupler 122 can be a lumen defined by the main shaft 120 through which the guidewire GW1 can be disposed and such that the main shaft 120 can be slidably disposed about the guidewire GW1. The guidewire GW1 can be any suitable size. In some embodiments, for example, the guidewire GW1 can have a diameter of about 0.014 inches to about 0.035 inches in diameter. In some embodiments, the guidewire GW1 can be about 0.025 inches diameter. With the guidewire coupler 122 movably coupled to the delivered guidewire GW1, the septum puncture device 100 can be advanced along the guidewire GW1 into the heart, as shown in FIG. 2A. More specifically, with the main shaft 120 coupled to (1) the body 110 and (2) the side catheter guide 130 via the guide coupler 140, the body 110, the main shaft 120, the guide coupler 140, the side catheter guide 130, the side catheter 160, the septum penetrator 170, and the guidewire coupler 172 all can be advanced into the heart of the patient as shown in FIG. 2A, such that body 110 extends through the IVC and into the RA, and the main shaft 120 extends into the SVC. With the main shaft 120 spanning the IVC, RA, and SVC, the main shaft 120 can provide a foundation or backstop against which the side catheter guide 130, side catheter 160, and septum penetrator 170 can be deployed and advanced towards the septum, as described in further detail herein.

In some instances, a distal end of the (1) main shaft 120, (2) side catheter guide 130, (3) side catheter 160, and septum penetrator 170 (and accompanying couplers, e.g., the guidewire coupler 122 and the guidewire coupler 172), can be disposed within the body 110 (e.g., within one or more lumens (not shown) defined by the body 110). In this manner, during delivery, the patient's anatomy can be protected or shielded by the body 110 to avoid inadvertent trauma to or contact with the patient's anatomy from such components. With a distal end of the body 110 disposed in or near the RA, the body 110 can be withdrawn (and/or one or more of the components movably coupled thereto can be advanced), thereby exposing the side catheter guide 130 and guide coupler 140 within the RA.

With the side catheter guide 130 exposed within the RA and translationally fixedly coupled to the main shaft 120 via the guide coupler 140, the side catheter guide 130 can be actuated to laterally deflect the distal end of the side catheter guide 130 (and as a result, also the side catheter 160, the septum penetrator 170, and the guidewire GW2 if disposed in the side catheter guide 130 during its lateral deflection), as shown in FIG. 2B. The side catheter guide 130 can be laterally deflected at any angle suitable to direct the side catheter 160 and septum penetrator 170, which are movably attached to the side catheter guide 130, towards the target penetration site, e.g., the FO, as shown in FIG. 2B. In some instances, an optimal angle of entry to the FO is 90 degrees or substantially 90 degrees relative to a surface line tangent to the FO, which can be about a similar angle relative to a central axis of the main shaft 120. Such a perpendicular (or substantially perpendicular) angle of entry can minimize the force required to penetrate the FO because the entire or substantially entire force vector is directed at the plane of the FO (rather than a tangential approach). Additionally, such a perpendicular (or substantially perpendicular) angle of entry, given the nature of a patient's anatomy, directs the septum penetrator 170 to a relatively large open space within the LA, thereby minimizing risk of inadvertent puncture within the LA (e.g., inadvertent puncture of a wall of the LA).

In other instances, the angle of entry relative to the FO or relative to the central axis of the main shaft 120 can be anywhere within a range of about 50 degrees to about 90 degrees. In some instances, the preferred angle of entry can be selected based on a particular therapy planned for the left side of the heart. The angle of entry, for example, defines the trajectory for the subsequent therapeutic device to enter the left side of the heart, and so in some instances an optimal angle and location of entry through the FO is based on a particular therapeutic device or procedure.

Note that the guidewire GW2 can be delivered in any suitable manner. In some instances, for example, the guidewire GW2 is disposed within the side catheter guide 130 during delivery of the side catheter guide 130, while in other instances the guidewire GW2 is inserted at a later time during the procedure, e.g., after the septum penetrator 170 has penetrated the FO and reached the LA.

With the side catheter guide 130 transitioned to its deployed configuration, in which the side catheter guide 130 is laterally deflected towards the FO, the side catheter actuator 164 can be actuated to advance the side catheter 160 along a path defined at least in part by the side catheter guide 130 and towards the FO. In some instances, the side catheter 160 is advanced until it's distal end tents or otherwise contacts the FO. For embodiments that include the end effector 162, the side catheter 160 can be advanced until the end effector 162 extending from the distal end of the side catheter 160 tents or otherwise contacts the FO.

In embodiments in which the end effector 162 is expandable and compressible, the end effector 162 can be delivered to the Right Atrium RA in a compressed or relatively small configuration, and then transitioned to a deployed configuration in which the end effector 162 is expanded to a relatively larger configuration, and then advanced to engage with the FO. After sufficient penetration of the Atrial Septum AS with the septum penetrator 170, as described in further detail herein, the end effector 162 can be transitioned to its retracted or compressed configuration suitable to be withdrawn from the patient. In embodiments in which the side catheter 160 is slidably disposed within a lumen defined by the side catheter guide 130, the end effector 162 can similarly be slidably disposed within the lumen defined by the side catheter guide 130 such that the side catheter guide 130 contains the end effector 162 in its constrained or compressed configuration during delivery, and then as the side catheter actuator 164 is actuated to advance the side catheter 160 distally from the distal end of the side catheter guide 130, the end effector 162 can transition to its expanded or unconstrained configuration as or after it exits the lumen of the side catheter guide 130.

With the side catheter 160 (or end effector 162) in sufficient contact with the FO, the penetrator actuator 174 can be actuated to advance the septum penetrator 170 relative to and along a path defined at least in part by the side catheter 160. The septum penetrator 170 can be advanced through the FO and across the Atrial Septum AS and into the Left Atrium LA. In some embodiments, the side catheter 160 defines a lumen through which the septum penetrator 170 is slidably disposed such that actuating the penetrator actuator 174 advances the septum penetrator 170 through the lumen of the side catheter 160. The septum penetrator 170 can be advanced in this manner to penetrate the FO and to extend into the left atrium LA. During such penetration, the main shaft 120 can provide lateral or axial stability to the septum penetrator 170.

As the distal end of the septum penetrator 170 is advanced across the Atrial Septum AS and into the Left Atrium LA, the guidewire GW2 can follow via the guidewire coupler 172 and the septum penetrator 170 in instances in which the guidewire GW2 is coupled to the side catheter guide 130 during delivery of the side catheter guide 130. In other instances, the guidewire GW2 can be inserted at a later time during the procedure, e.g., after the septum penetrator 170 has penetrated the FO and reached the LA In some embodiments, the guidewire coupler 172 is a lumen defined by the septum penetrator 170 and through which the guidewire GW2 can be slidable disposed. In such embodiments, the guidewire GW2 can be disposed within the lumen of the septum penetrator 170 during delivery and deployment of the septum penetrator 170 into the Left Atrium LA.

With the septum penetrator 170 and the guidewire GW2 disposed within the Left Atrium LA, the guidewire GW2 can be further advanced into the Left Atrium LA by manipulation of the guidewire GW2 at its proximal end, and/or the septum penetrator 170 can be withdrawn from the Left Atrium LA, across the puncture or entry site of the FO, leaving the guidewire GW2 within the Left Atrium LA.

With the guidewire GW2 delivered to the Left Atrium LA, and extending proximally from the Left Atrium LA across the puncture or entry site of the FO, into the Right Atrium RA, the IVC, and through the vasculature of the patient to the entry point of the patient (for subsequent access to the Left Atrium AS), the septum puncture device 100 can be withdrawn from the heart proximally over guidewire GW2 and from the patient.

The guidewire GW2 can be any guidewire suitable to provide desirable subsequent access to the Left Atrium LA. In some embodiments, for example, the guidewire GW2 can be a pigtail, atraumatic guidewire or other suitable guidewire conventionally used in transseptal procedures. For example, the guidewire GW2 can have a flexible, spiral tip, pigtail, and can be configured to anchor the septum puncture device 100 to the LA, thereby limiting or preventing the guidewire GW2 from being inadvertently withdrawn or removed from the LA in response to or while the septum puncture device 100 is being withdrawn along the guidewire GW2 and from the patient. Another example guide GW2 can be a ProTrack™ Pigtail Wire from Baylis Medical Company, Inc.

The septum puncture device 100 can be configured to be withdrawn from the patient in any suitable sequence (e.g., after the guidewire GW2 has been delivered to the Left Atrium LA). With the guidewire GW2 disposed within the Left Atrium LA, for example, the portions of the septum penetrator 170 and guidewire coupler 172 disposed within the Left Atrium LA can be withdrawn relative to the guidewire GW2 and through the puncture site in the FO and into the Right Atrium RA. In embodiments in which the side catheter 160 defines a lumen through which the septum penetrator is slidably disposed, the septum penetrator 170 can be withdrawn relative to and into the lumen defined by the side catheter 160. In this manner, the septum penetrator 170, and particular it's distal that is designed to penetrate tissue, can be sheathed or shielded by the side catheter 160 to facilitate safe withdrawal from the patient and avoid inadvertent contact with the patient's heart or vasculature during removal of the septum puncture device 100 from the patient.

Similarly, the side catheter 160 can be withdrawn relative to the side catheter guide 130. For example, in embodiments in which the side catheter guide 130 defines a lumen through which the side catheter 160 is slidably disposed, the side catheter 160 can be withdrawn into the lumen of the side catheter guide 130. In embodiments in which the septum puncture device 100 includes an end effector 162, the side catheter guide 160 can be withdrawn relative to and into the lumen of the side catheter guide 130 such that the end effector 162 is also withdrawn into the lumen of the side catheter guide 130. In embodiments in which the end effector 162 has a deployed configuration with a diameter larger than an internal diameter of the side catheter guide 130, the end effector 162 can be configured to be transitioned from its deployed configuration to its withdrawal (or delivery) configuration. For example, if the end effector 162 is a balloon, it can be deflated and then withdrawn into the lumen of the side catheter guide 130. As another example, if the end effector 162 includes or is formed of shape memory material, the end effector 162 can be compressed, constrained, or otherwise transitioned to a smaller arrangement such that it can be withdrawn into the side catheter guide 130. In some instances, withdrawal of the end effector 162 into the side catheter guide 130 can cause the end effector 162 to transition to its constrained or compressed configuration.

Further, the side catheter guide 130 can be configured to transition from its deployed configuration in which its distal portion is laterally deflected relative to the main shaft 120 to its withdrawal (or delivery) configuration in which the side catheter guide 130 is at least substantially linear and parallel to the main shaft 120. In some embodiments, for example, a proximal force can be applied to a proximal end portion of the side catheter guide 130 to withdraw the side catheter guide 130 relative to the main shaft.

With the septum puncture device 100 disposed as shown in FIG. 2A, for example, after delivering the guidewire GW2, the septum puncture device 100 can be withdrawn from the heart and from the patient. For example, the body 110, and all of the components coupled thereto, can be withdrawn from the heart, through the patient's vasculature, and out through the initial entry site into the patient (e.g., the femoral puncture site).

Although embodiments described herein refer to introducing a guidewire and septum puncture device into the patient's vasculature, and across the IVC and RA, and into the SVC, access to the RA for purposes of deploying a septum penetrator, can be accomplish in a variety of ways. In some embodiments, for example, the guidewire and septum puncture device can be inserted into a patient's jugular vein (e.g., right internal jugular vein), and then advanced into and across the SVC and RA, and into the IVC, such that a distal end of the septum puncture device is disposed in the IVC (or beyond).

Although embodiments described herein refer to a single FO puncture to deliver a single guidewire to the LA, it should be understood that the septum puncture devices described herein can be used to perform multiple punctures and to deliver multiple guidewires. In some instances, for example, a double puncture and delivery of two guidewires may be desirable, e.g., in connection with an atrial fibrillation ablation procedure. In such instances, the septum puncture devices described herein can be deployed twice to puncture the septum twice, with each puncture providing access to deliver a guidewire, as described herein. In some procedures that require multiple punctures and guidewires delivered to the LA, for example, it can be crucial that the punctures are in a particular location and located a particular distance from each other, and as described through this disclosure, the septum puncture devices described herein provide just that.

Further, instead of using a septum puncture device described herein to administer multiple punctures in series (e.g., with a single penetrator, single side catheter, single side catheter guide, etc.), in some embodiments, any of the septum puncture devices described herein can be modified to incorporate additional components. For example, in some instances, a septum puncture device can include a body and a main shaft (similar to septum puncture device 100), but also include two side catheter guides, two side catheters, two end effectors, two septum penetrators, and two guide couplers (for the guidewires being delivered), and optionally one or two guide couplers and one or two guide stabilizer/actuators. In this manner, two side catheter guides can be deployed (i.e., laterally deflected and stabilized) simultaneously, and then two side catheters (optionally with end effectors) can be advanced, optionally simultaneously, to contact the septum, and then two septum penetrators can be advanced, optionally simultaneously, to penetrate the septum. With two punctures in the septum, two guidewires can then be delivered, optionally simultaneously. In such instances, the preferred distance between the two punctures can be selectively defined by the distance between the side catheters from which the septum penetrators are advanced.

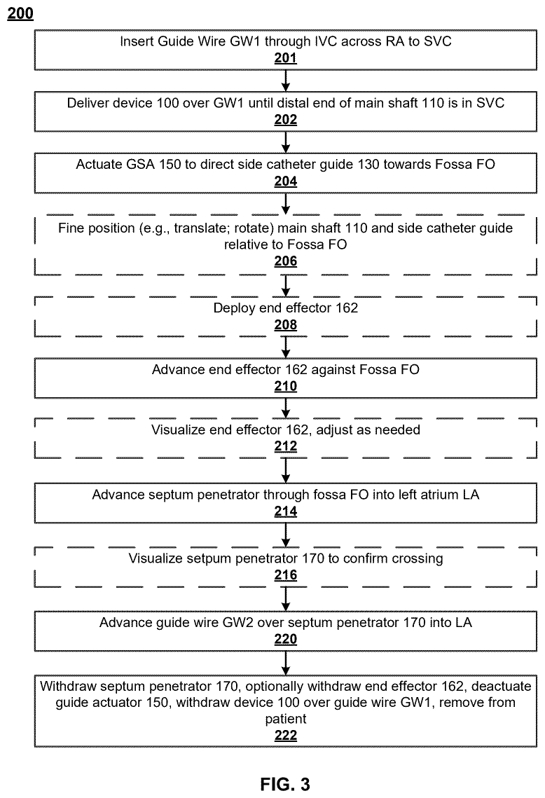

FIG. 3 illustrates a method 200 of using the septal puncture device 100 to access a left atrium of a heart of a patient, according to an embodiment. At 201, the guidewire GW1 is inserted through the IVC, across the RA, and into SVC of the heart (e.g., via a femoral vein puncture and through the patient's vasculature disposed between the femoral vein puncture site and the IVC). At 202, the septal puncture device 100 is delivered over the guidewire GW1 until a distal end of a main shaft 110 is disposed within the SVC. At 204, the GSA 150 is actuated to laterally deflect and direct the side catheter guide 130 towards the FO. Optionally, at 206, the main shaft 110 and the side catheter guide 130 are selectively positioned (e.g., translated or rotated) relative to the FO. Optionally, at 208, the end effector 162 is deployed. At 210, with the end effector 162 deployed, the end effector 162 can be advanced towards and into contact with the FO to tent the FO. To advance the end effector 162 towards and into contact with the FO, the side catheter 160 can be advanced (e.g., by actuating the side catheter actuator, not shown) relative to the side catheter guide 130) or by manipulating (i.e., translating or rotating) the main shaft 120. Optionally, at 212, the end effector 162 (or distal end of side catheter 130) is visualized from outside the patient. As described elsewhere herein, both the end effector and the tenting of the FO (or other portion of the septum) are visible to the operator from outside the patient via various imaging technologies, such as, for example, ultrasound or related suitable imaging technologies. If necessary, the main shaft 110 or the side catheter guide 130 are adjusted to selectively reposition the end effector 162 (or distal end of side catheter 130) relative to the FO. In instances in which the operator is not satisfied with the location on the septum contacted or tented by the end effector 162, e.g., if the end effector 162 is misaligned with the FO, the end effector 162 can be withdrawn from contact with the FO or septum (e.g., by withdrawing the side catheter 160 relative to the side catheter guide 130 or by manipulating the main shaft 120), and then the operator can make another approach at landing the end effector 162 on the FO in a manner sufficient for subsequent puncturing of the FO. This process can be repeated until the operator is satisfied.

At 214, the septum penetrator 170 is advanced through the FO and into the LA. Optionally, at 216, visualization techniques are used to confirm crossing of the septum penetrator 170 into the LA. At 220, the guidewire GW2 is advanced relative to the septum penetrator 170 and into the LA or the septum penetrator 170 is withdrawn relative to the septum penetrator 170, thereby leaving a portion of the guidewire GW2 in the LA. At 222, the septum penetrator 170 is withdrawn, the end effector 162 is optionally withdrawn, the main shaft 120 is withdrawn, the guide actuator 150 is deactuated, and the device 100 is withdrawn over the guidewire GW1 and removed from the patient.

Although not shown, in some embodiments, any of the main shafts described herein can define a channel through which an intra-cardiac echo can be disposed or slidably coupled to assist in navigation through the patient.

In some embodiments, a needle can be aimed at a specific region of the FO for puncture. The FO can be divided into quadrants, for example, in which a puncture in each quadrant is advantageous for a specific procedure. The needle can thereby be aimed to puncture slightly superior, posterior, and 3.5 cm-4.5 cm above the mitral valve for a MitraClip device, or to puncture posterior and slightly inferior within the FO for typical left atrial appendage occlusion devices. After successful puncture and insertion of a guidewire, the septum puncture device can be completely removed to make way for any suitable instrument or device to be guided into the left atrium of the heart to perform a desired procedure, such as atrial fibrillation ablation, left atrial appendage closure, and valve replacements.

In some cases, a septum puncture device can be configured for electrophysiological (EP) procedures. In EP procedures, in some instances, it may be desirable to reduce the size of the septum puncture device. Common transseptal sheaths used for EP procedures, for example, often have an 11 F outer diameter and about an 8.5-9 F inner diameter. Accordingly, to accommodate such procedures, a septum puncture device can be correspondingly sized, e.g., to have an outer diameter around or no larger than an associated transseptal sheath.

Devices described above provide several clinical benefits, including providing the operator with the ability to independently adjust the angle of approach to the septum and lateral displacement to contact and tent the fossa. In some instances, it may be desirable to access the left atrium for EP procedures using existing steerable sheath technology, but in a unique way (a stacked arrangement, as described in further detail herein) to provide the operator with increased degree of control and independent degrees of freedom, similar to as described in various embodiments herein.

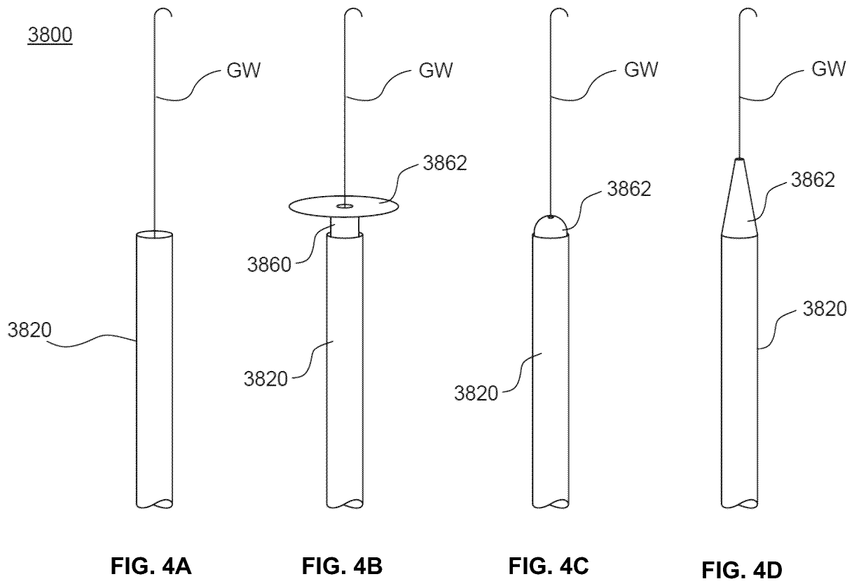

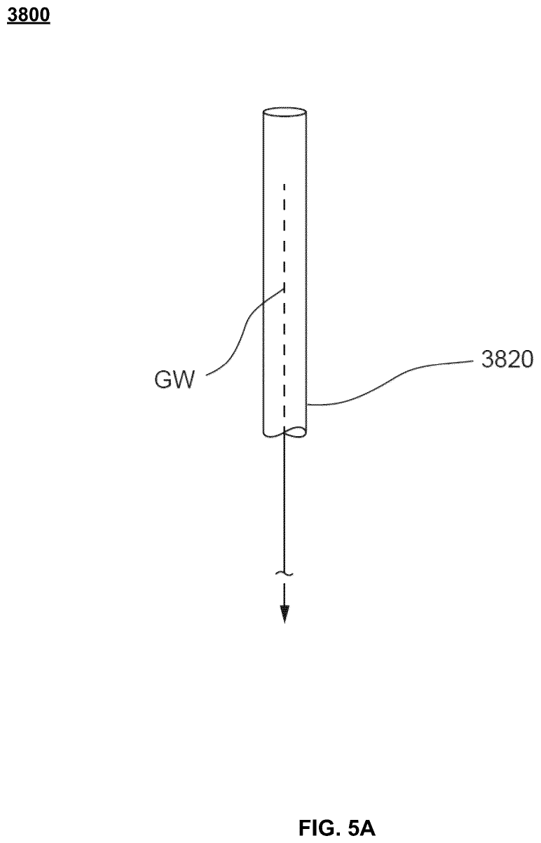

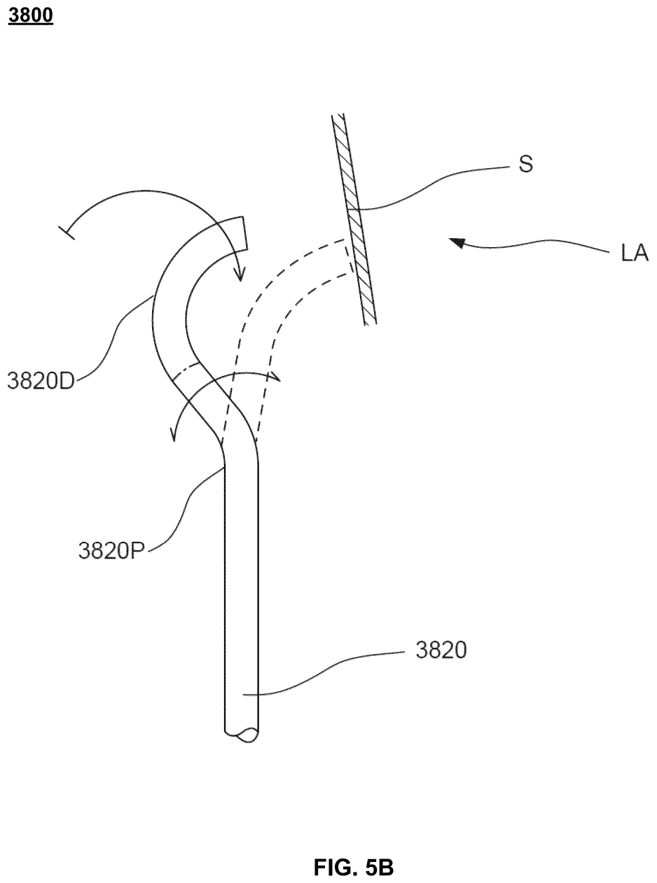

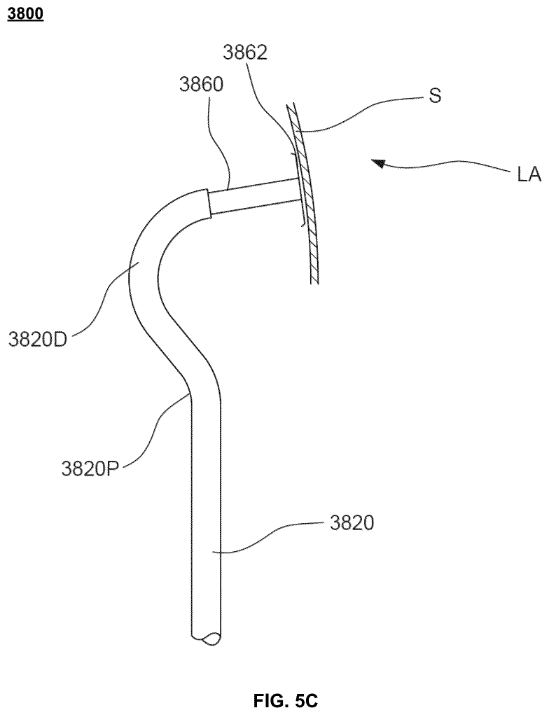

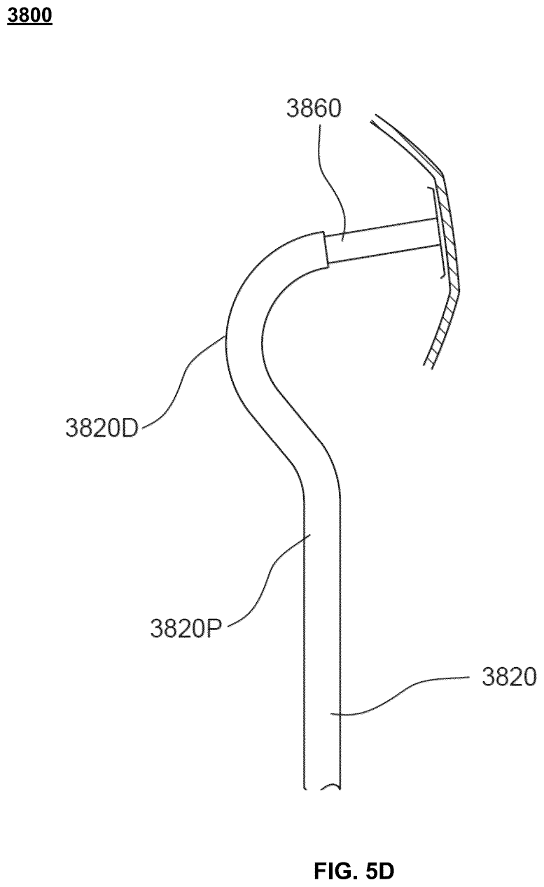

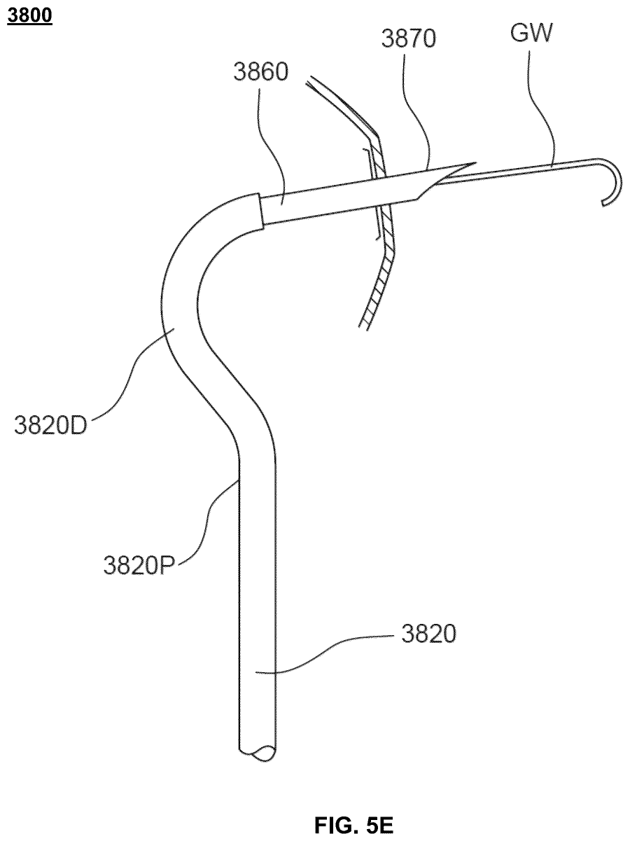

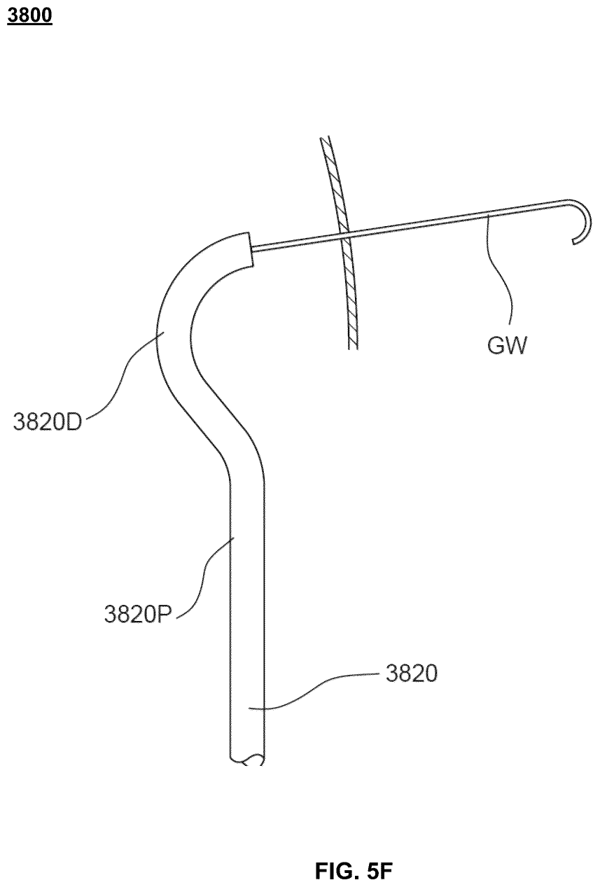

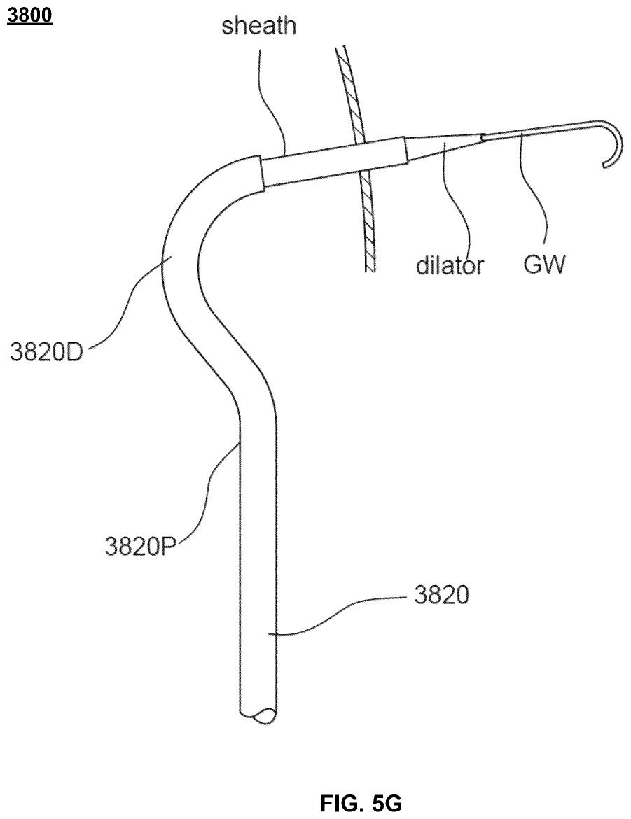

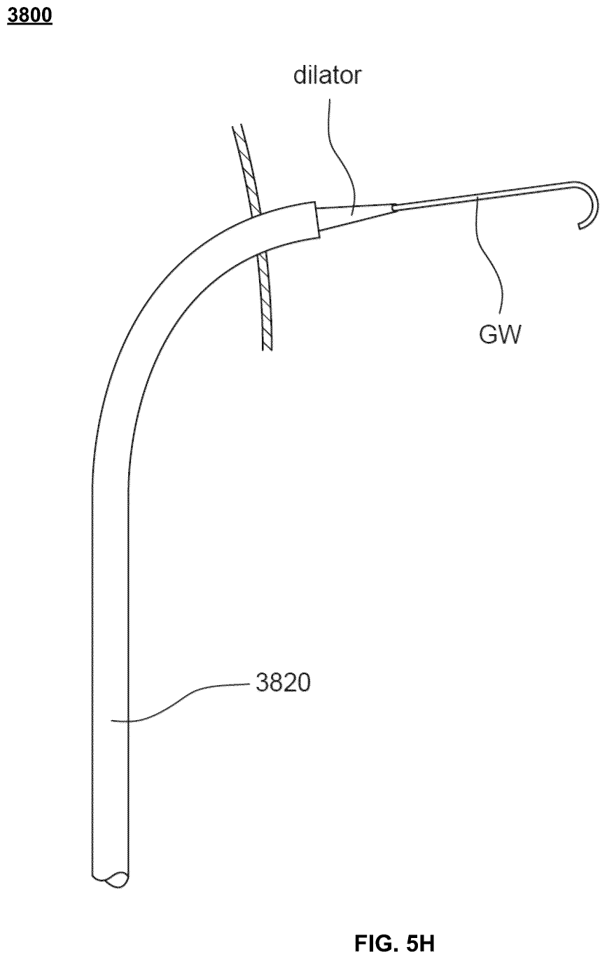

FIGS. 4A-4D illustrate such a device 3800, with slight variations. FIG. 4A illustrates device 3800 having a steerable catheter 3820 disposed over a guidewire GW. FIG. 4B illustrates the catheter 3820 with a catheter disposed therein and having an end effector at its distal end. FIG. 4C illustrates the catheter 3820 with the catheter having a blunt-nosed atraumatic end at its distal end. FIG. 4D illustrates the catheter 3820 with the catheter having a tapered dilator at its distal end. The following description in accordance with FIGS. 5A-5H illustrate an example delivery and deployment sequence of device 3800. Similar to or the same as described with respect to other septum puncture devices described herein, the septum puncture device 3800 can be used to access a left side of the heart (e.g., left atrium) from the right side of the heart (e.g., right atrium) and to deliver a guidewire to the left side of the heart. The septum puncture device 3800 can be constructed the same as or similar to, and can function the same as or similar to, any of the septum puncture device described herein. Thus, portions of the septum puncture device 3800 are not described in further detail herein.

In this embodiment, to accommodate the desired access size for EP or IC procedures, septum puncture device 3800 can have an outer diameter between about 10 F and about 14 F, and in some instances, between about 11 F and about 12 F. In some embodiments, the catheter 3820 may not be steerable. In some embodiments, the sheath 3820 may be semi-rigid and may be provided with a predetermined shape (e.g., a predetermined curve). In some embodiments, the sheath 3820 may be configured such that an operator can manipulate the shape of at least a portion of the sheath 3820 (e.g., via manual adjustment, heat setting, and/or any other suitable method) before the procedure and/or during the procedure. In some embodiments, the sheath 3820 may be shapeable such that the user may remove the sheath 3820 from the body, adjust the shape of the sheath 3820, and then re-insert the sheath 3820 into the body. In some embodiments, the sheath 3820 may be steerable with one or more pull-wires to adjust an angle of at least a portion of the sheath 3820. In some embodiments, the sheath 3820 may be provided in a predetermined non-linear shape suitable for reaching the septum for typical patient population, but can be adjustable prior to and/or during the procedure by the operator to adjust to a particular patient's anatomy.

As shown in FIG. 5A, device 3800 includes the catheter 3820 shown with a guidewire GW disposed therein. Although not shown, a dilator may be disposed around the guidewire GW and inside the catheter 3820. As shown, the guidewire GW is retracted into the catheter 3820 to accommodate maneuvering and engagement of device 3800 with the fossa. As described in more detail herein, the guidewire GW can be retained within the catheter 3820 and selectably extended to function as a transseptal guidewire in subsequent steps/procedures.

FIG. 5A illustrates the device 3800 in a delivery configuration (e.g., substantially straight), suitable to insert the catheter 3820 through the IVC about the guidewire GW for atraumatic passage therethrough. Similar to the steps described with respect to device 3700, in this embodiment, the guidewire GW can be disposed within the innermost lumen of the device 3800, i.e., the lumen of the septum penetrator 3870. The device 3800 can be delivered over the guidewire GW that has already been placed in SVC, or in some instances, the device 3800 can be delivered with only a small portion of the guidewire GW extending from a distal end portion thereof.

The catheter 3820 includes a distal section 3820D and a proximal section 3820P, each of which are independently adjustable, to yield a desired combination of angle and lateral displacement, as shown in FIG. 5B. In some implementations, the distal 3820D and proximal sections 3820P can be formed separately and then combined (e.g., in a stacked arrangement), while in some implementations, the distal 3820D and proximal sections 3820P can be monolithic, but having independent controls (e.g., each having independent control wires operably coupled thereto).

In some implementations, the distal section 3820D is configured to be deflectable in only one direction, e.g., from nominally straight to fully flexed, such as nominally 90 degrees, while the proximal section 3820P may be bi-directional, (e.g., from about −180 degrees, nominally straight, to about 180 degrees). In some embodiments, the proximal section 2830P may be bi-directionally deflectable between about −45 degrees, nominally straight, and 45 degrees. This combination allows the operator to select a direction (e.g., to be orthogonal to the plane of the fossa), while independently also setting a lateral deflection, ranging from about zero (e.g., residing on the long-axis of the device) to a maximum distance. Optionally, in some implementations, a distal end of the distal section 3820 may be advanced to contact the fossa, e.g., without any extendable catheter disposed therein and/or an end effector, as illustrated in dashed lines in FIG. 5B.

In some implementations, a side catheter 3860 with end effector 3862 at its distal end can be disposed with the lumen of the catheter 3820, and extended towards the fossa, as shown in FIG. 5C, and similar to as described in various embodiments herein. The end effector 3862 can be of any suitable form, for example, similar to or the same as any of the end effectors described herein, and/or any of the arrangements illustrated in FIGS. 4A-74D, for example.

In some implementations, a distal most element of device 3800 (e.g., a distal end of the side catheter 3860 and/or the end effector 3862) may be formed of echogenic materials, constructions, and/or include echogenic attachments and/or coatings, coverings, etc., thereby providing an indication on echocardiography (e.g., TEE or ICE) showing the operator the location of the device end in relation to the fossa.

The optional extendable side catheter 3860 is shown in FIG. 5D extended (with end effector 3860 disposed at its distal end) into contact with the fossa. Similar to as described in various embodiments, the side catheter 3860 and end effector 3860 can be advanced to tent the fossa.

With the fossa tented, the septum penetrator 3870 (e.g., a mechanical needle) can be advanced through the lumen of the catheter 3820 to pierce the septum and enter the left atrium, followed by advancement and delivery of the guidewire GW to the left atrium, as shown in FIG. 5E.

Subsequently, the septum penetrator 3870 can be withdrawn from the left atrium and into the catheter 3820, leaving the guidewire GW extended into the left atrium, as shown in FIG. 5F. Similar to as described in connection with device 3700, with the guidewire GW traversing the fossa and extending into the left atrium, the device 3800 can be withdrawn and removed from the patient, leaving the guidewire GW in place to be used to delivery sheath and/or sheath/dilator combinations across the fossa and into the left atrium, thereby providing access to the left atrium for therapeutic EP devices.

Additionally, or alternatively, device 3800 can be sized (e.g., sized with a particular inner diameter) to accept a dilator/sheath combination through its lumen after the septum penetrator 3870 and any nested components (e.g., side catheter 3860) have been withdrawn from the catheter 3820. In this manner, the device 3800 and its curvature provide support for the dilator/sheath to guide it along the transseptal guidewire GW directly to the crossing point and aid the dilation of the fossa puncture, as illustrated in FIG. 5G. After remove of the guidewire GW and dilator, for example, the transeptal sheath can provide access to the left atrium for therapeutic EP devices.

Additionally, or alternatively, device 3800 can be sized (e.g., sized with a particular inner diameter) to accept a dilator through its lumen after the septum penetrator 3870 and any nested components (e.g., side catheter 3860) are withdrawn and removed from the patient, as shown in FIG. 5H. The device 3800 and its curvature provide support for the dilator to guide it along the guidewire GW directly to the crossing point and aid the dilation of the fossa. Adjustment of the proximal 3820P and distal 3820D sections can be used to cross the fossa with the dilator, followed by the distal end of the catheter 3820. After removal of the guidewire GW and dilator, the catheter 3820 can itself provide transseptal access to the left atrium for therapeutic EP devices, and/or the like.

In may also be advantageous to incorporate one or more dilators into a catheter, such as in the device 3800 described above. Such an alternative catheter design is described below.

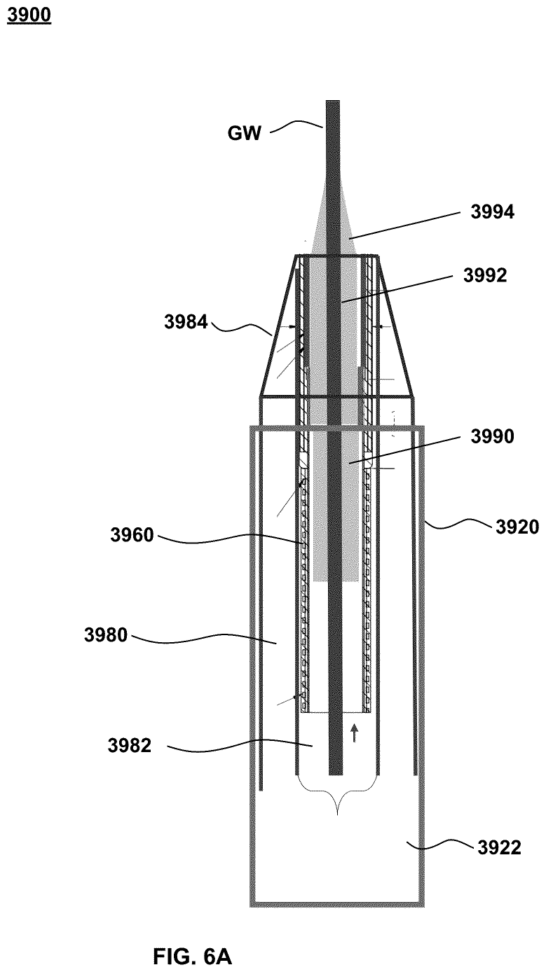

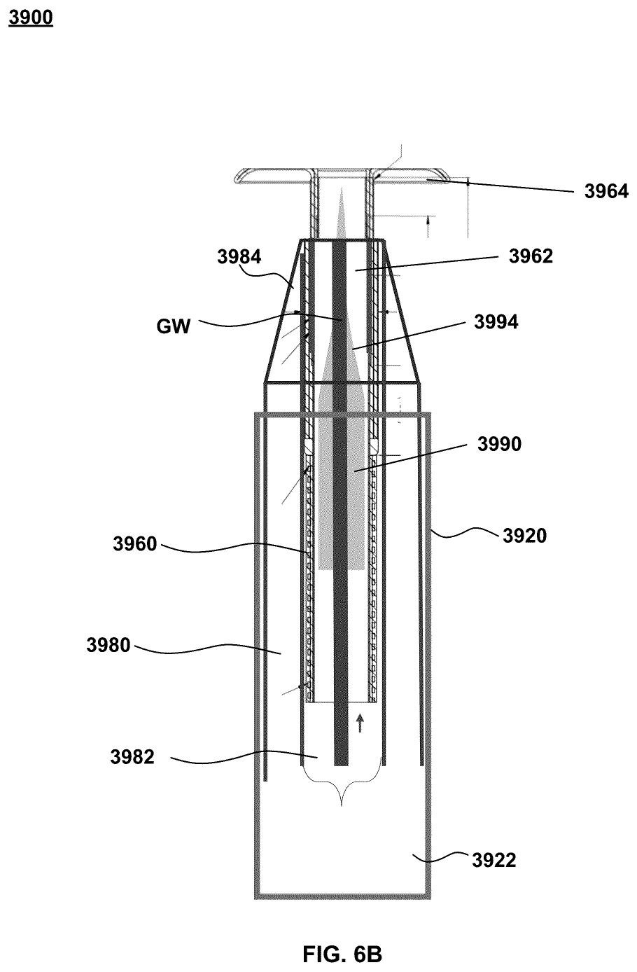

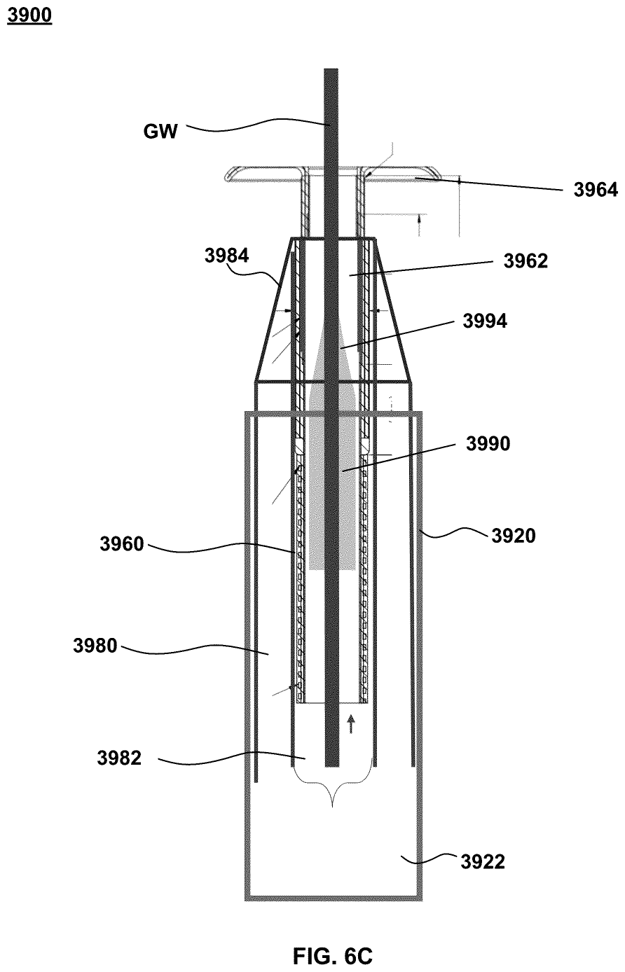

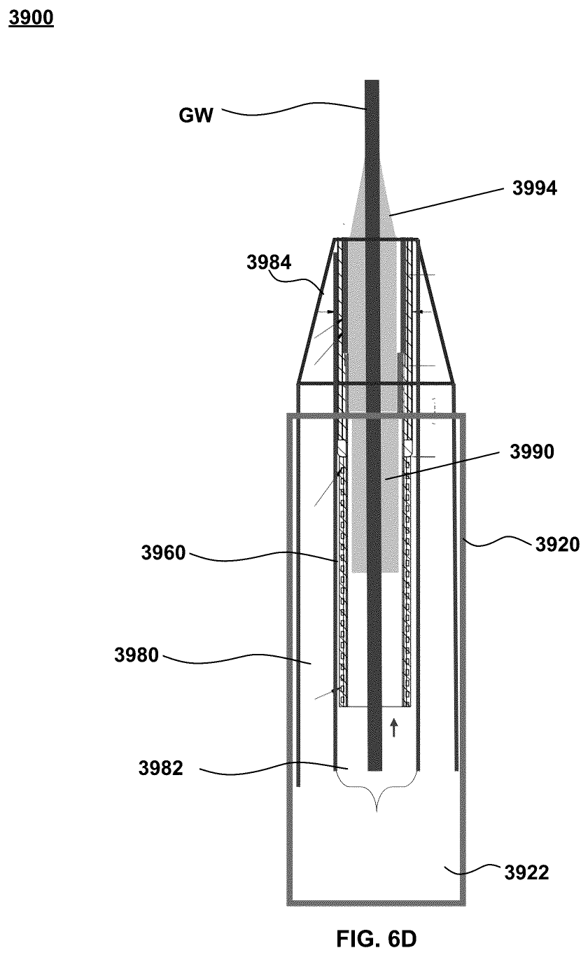

FIGS. 6A-6D schematically illustrate such a device, 3900. The following description in accordance with FIGS. 6A-6D illustrate the components and an example delivery and deployment sequence of device 3900. Similar to or the same as described with respect to other septum puncture devices described herein, the septum puncture device 3900 can be used to access a left side of the heart (e.g., left atrium LA) from the right side of the heart (e.g., right atrium RA) and to deliver a guidewire to the left side of the heart. In some embodiments, as described in more detail below, the device 3900 can also be used to deliver the therapy device to the left atrium LA. The septum puncture device 3900 can be constructed the same as or similar to, and can function the same as or similar to, any of the septum puncture device described herein. Thus, portions of the septum puncture device 3900 are not described in further detail herein.

In this embodiment, to accommodate the desired access size for EP procedures, septum puncture device 3900 can have an outer diameter between about 6 F and about 14 F, and in some instances, between about 11 F and about 12 F. For use in interventional cardiology (“IC”) applications, device 3900 can have a substantially larger outer diameter, to enable delivery of larger therapy devices, such as 14-16 F (e.g., for a left atrial appendage occlusion (LAAO) device), 22-24 F (e.g., for a MitraClip steerable guide), or up to 35 F (e.g., for a transcatheter mitral valve replacement “TMVR”) device).

As shown in FIGS. 6A-6D, device 3900 includes a shapeable (e.g., steerable or pre-shaped) catheter (also referred to as a sheath) 3920, an outer dilator 3980, an inner dilator 3990, and a side catheter (also referred to as an inner guide catheter) 3960. In some embodiments, the catheter 3920 may be substantially similar to the catheter 3820. Catheter 3920 has an internal lumen 3922, and can be constructed and operated in the same manner as side catheter 3820 described above. Outer dilator 3980 has an internal lumen 3982 and a tapered tip 3984 at its distal end, and can be slidably disposed with the inner lumen 3922 of the catheter 3920. In turn, inner guide catheter 3960 has an internal lumen 3962 and an end effector 3964 at its distal end, and can be slidably disposed with the inner lumen 3982 of the outer dilator 3980. In turn, inner dilator 3990 has an internal lumen 3992 and a tapered tip 3994 at its distal end, and can be slidably disposed with the inner lumen 3962 of the inner guide catheter 3960. A guidewire GW can be slidably disposed in inner lumen 3992 of inner dilator 3990. The inner guide catheter 3960 (and end effector 3964), the outer dilator 3980, and the inner dilator 3990 can be decoupled from catheter 3920, so that they can be withdrawn from the inner lumen 3922 of catheter 3920 to enable exchangeless delivery of a therapy device to the left atrium LA through catheter 3920.

In this embodiment, as discussed in connection with other embodiments above, guidewire GW may be an RF guidewire, for example a VersaCross® RF guidewire available from Baylis Medical. Thus, guidewire GW may serve the same function as the septum penetrator (such as septum penetrator 170) but is solid-tipped and can be electrified with radiofrequency energy to puncture the FO. Guidewire GW can also serve the same function as the guidewire delivered through the septum penetrator in previous embodiments, e.g., to provide a guide over which therapeutic devices can be delivered into the right atrium RA, and can also serve as a guide over which the device 3900 is delivered into the left atrium LA. As shown, the guidewire GW can be retracted into the catheter 3920 to accommodate maneuvering and engagement of device 3900 with the fossa. As described in more detail herein, the guidewire GW can be retained within the catheter 3920 and selectably extended to function as a septum penetrator and as a transseptal guidewire in subsequent steps/procedures.

In this embodiment, inner dilator 3990 can serve as a guide for delivery of puncture device over guidewire GW and to support guidewire GW when extended to puncture the septum. Its tapered tip 3994 can provide a transition from the outer diameter of guidewire GW to the inner diameter of outer dilator 3980, and tapered tip 3984 of outer dilator 3980 can provide a transition to the inner diameter of catheter 3920—these transitions can reduce trauma to the vasculature through which the device 3900 is delivered to the left atrium LA, and to dilate the puncture through the septum created by the guidewire GW to a diameter suitable for subsequent delivery of a therapeutic device through the inner lumen 3922 of catheter 3920 into the left atrium LA. In some embodiments, inner dilator 3990 can be omitted.

FIG. 6A illustrates the device 3900 in a delivery configuration (or insertion mode), suitable to insert the catheter 3920 through the IVC about the guidewire GW for atraumatic passage therethrough. Similar to the steps described with respect to device 3700, in this embodiment, the guidewire GW can be disposed within the innermost lumen of the device 3900, in this embodiment the lumen 3922 of the inner dilator 3990. The device 3900 can be delivered over the guidewire GW, which has already been placed in SVC, or in some instances, the device 3900 can be delivered with only a small portion of the guidewire GW extending from a distal end portion thereof.

When the device 3900 has been delivered over guidewire GW with its distal end in the right atrium, adjacent to the fossa, and its position/orientation adjusted so that the axis of the distal end is directed towards the fossa ((e.g., by bending/curving the catheter 3920, as described in more detail above in connection with device 3800), device 3900 can be reconfigured from the configuration shown in FIG. 6A to that shown in FIG. 6B. To do so, guidewire GW and inner dilator 3990 can be withdrawn proximally, and inner guide catheter 3960 can be extended distally, relative to outer dilator 3980. Upon distal extension of inner guide catheter 3960 (towards the fossa), end effector 3964 can self-expand into the configuration shown in FIG. 6B.

Similar to as described in various embodiments, the inner guide catheter 3960 and end effector 3960 can be advanced to probe and tent the fossa to confirm that the end effector 3964 is in contact with the desired location on the fossa.

As shown in FIG. 6C, the guidewire GW can then be extended through lumen 3992 of inner dilator 3990 into contact with the fossa, and can be energized with RF energy and pushed against and through the fossa, into the left atrium LA. Device 3900 can then be reconfigured from the configuration shown in FIG. 6C to that shown in FIG. 6D, by withdrawing inner guide catheter 3960 proximally relative to outer dilator 3980, drawing end effector 3964 into the lumen 3984 of outer dilator 3990, and by extending inner dilator 3990 through lumen 3962 of inner guide catheter 3960 so that tapered tip 3994 is distal to tapered tip 3984 of outer dilator 3980. In this configuration, device 3900 can be delivered distally over guidewire GW, through the fossa and into the left atrium LA.

Subsequently, the outer dilator 3980, inner guide catheter 3960, and inner dilator 3990 can be withdrawn proximally over guidewire GW through lumen 3922 of catheter 3920, and a therapeutic device can be delivered over guidewire GW through lumen 3922 of catheter, into the left atrium LA, as described above for device 3800. Alternatively, as also described above for device 3800, the entire device 3900 (including catheter 3920) can be withdrawn over guidewire GW, and a therapeutic device delivered over guidewire GW.

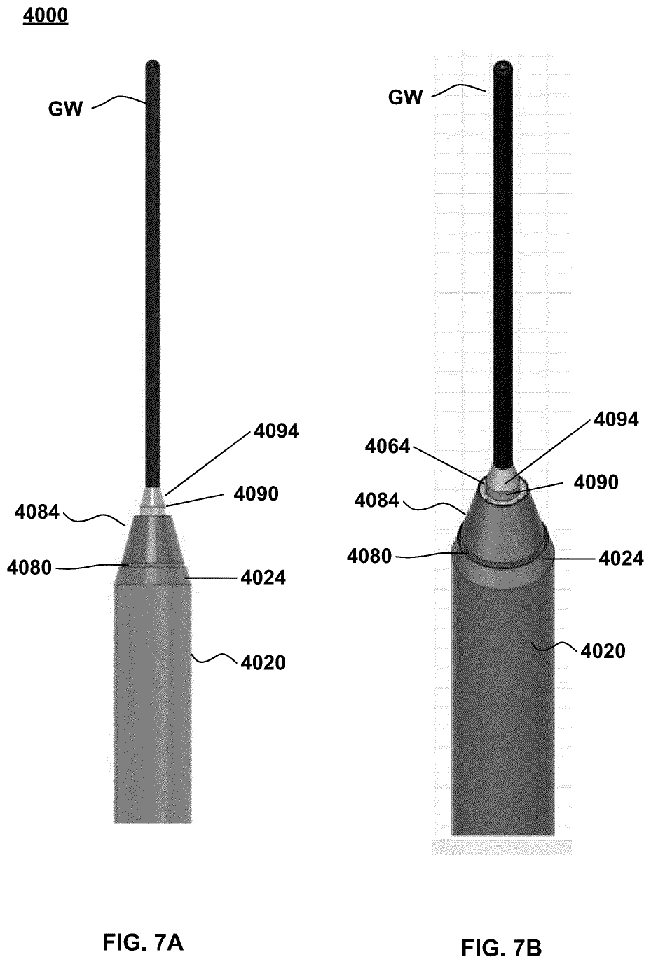

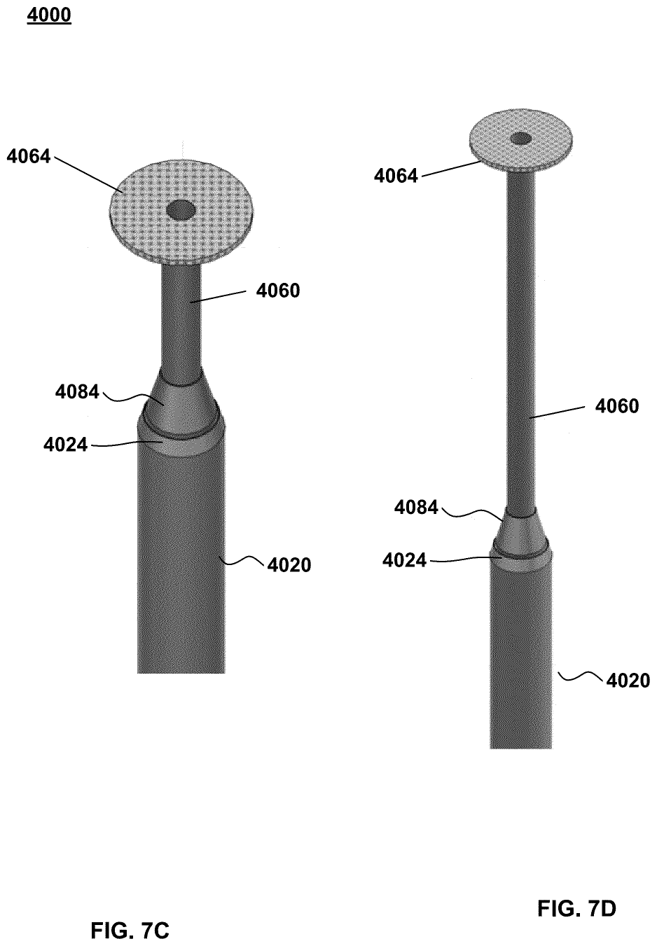

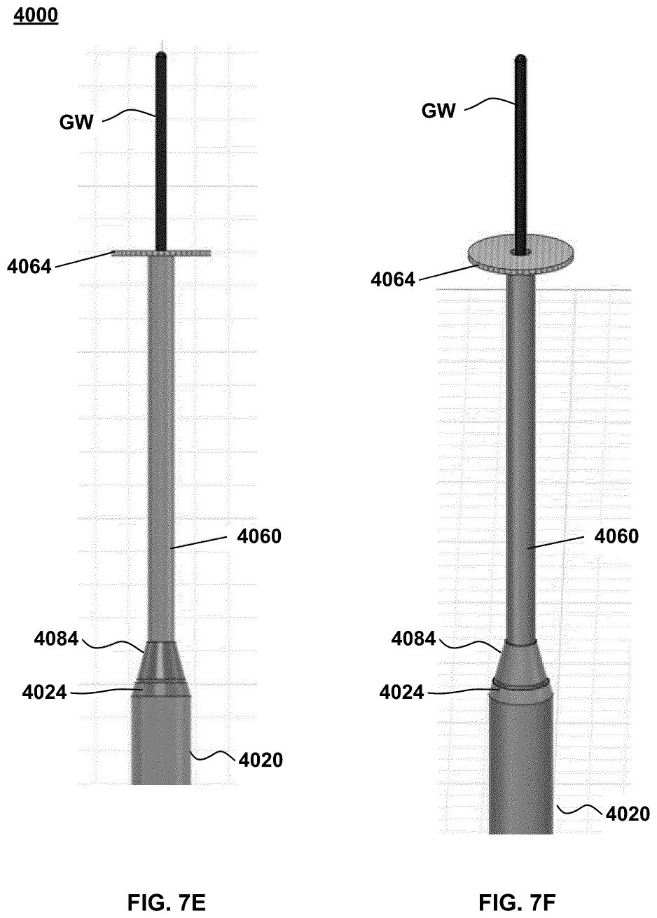

Another embodiment of a puncture device, 4000, is shown in FIGS. 7A to 7F. Puncture device 4000 is very similar to device 3900 and is therefore described briefly.

As shown in FIGS. 7A-7F, device 4000 includes a shapeable (e.g., steerable or pre-shaped) catheter (also referred to as a sheath) 4020, an outer dilator 4080, an inner dilator 4090, and an inner guide catheter 4060. In some embodiments, the catheter 4020 may be substantially similar to the catheters 3820, 3920. Catheter 4020 can be constructed and operated in the same manner as side catheter 3820 described above. In this embodiment, catheter 4020 includes a tapered tip 4024 at its distal end. Outer dilator 4080 has an inner lumen (not shown) and a tapered tip 4084 at its distal end, and can be disposed (e.g., fixedly disposed or slidable disposed) within an inner lumen (not shown) of the catheter 4020. In some embodiments, the outer dilator 4080 may be fixed inside the catheter 4020 but configured to be withdrawn. In some embodiments, the inner dilator 4090 may be slidably disposed through the catheter and configured to provide stiffness or rigidity to a portion of the device through which it extends. In turn, inner guide catheter 4060 has an internal lumen (not shown) and an end effector 4064 at its distal end, and can be slidably disposed with the inner lumen of the outer dilator 4080. In turn, inner dilator 4090 has an internal lumen (not shown) and a tapered tip 4094 at its distal end, and can be slidably disposed with the inner lumen of the inner guide catheter 4060. A guidewire GW can be slidably disposed in inner lumen of inner dilator 4090.

Device 4000 is shown in FIGS. 7A and 7B in its insertion mode (corresponding to FIG. 6A for device 3900), is shown in FIGS. 7C and 7D in a configuration corresponding to that shown in FIG. 6B for device 3900, and is shown in FIGS. 7E and 7F in a configuration corresponding to that shown in FIG. 6C for device 3900.

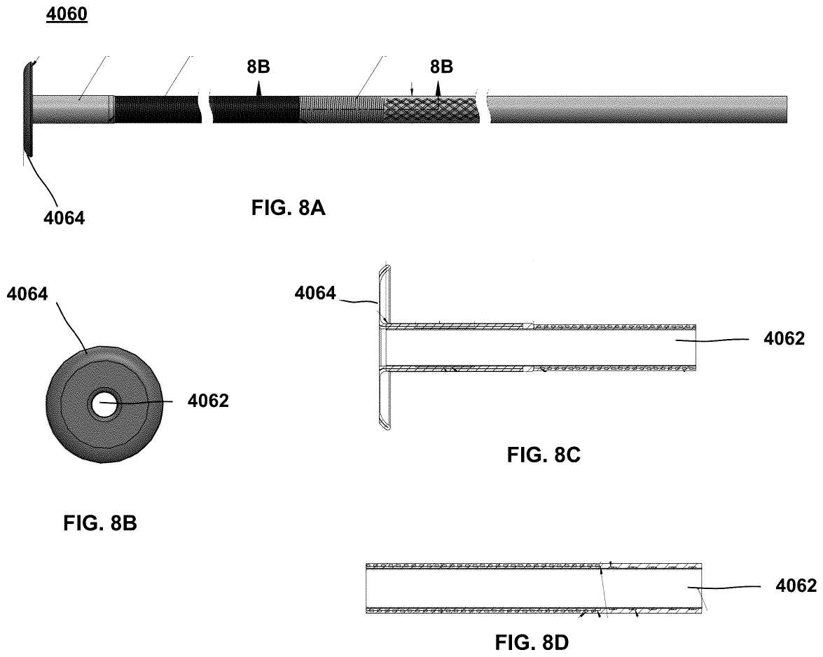

Inner guide catheter 4060 is shown in more detail in FIGS. 8A-8D. FIG. 8A is a side view of inner guide catheter 4060 in a deployed, unconstrained configuration, FIG. 8B is an end view of the inner guide catheter 4060, FIG. 8C is a longitudinal cross section through a distal portion of the inner guide catheter, and FIG. 8D is a longitudinal cross-section of the inner guide catheter along section 8B-8B of FIG. 8A. As described above, end effector 4064 is a self-expanding structure, and is collapsed when constrained within the inner lumen of the outer dilator 4080. As with the device 3900, for device 4000 the inner guide catheter 4060 (and end effector 4064) and the outer dilator 4080 (and, if present, an inner dilator) can be decoupled from catheter 4020, so that they can be withdrawn from the inner lumen 4022 of catheter 4020 to enable exchangeless delivery of a therapy device to the left atrium LA through catheter 4020.

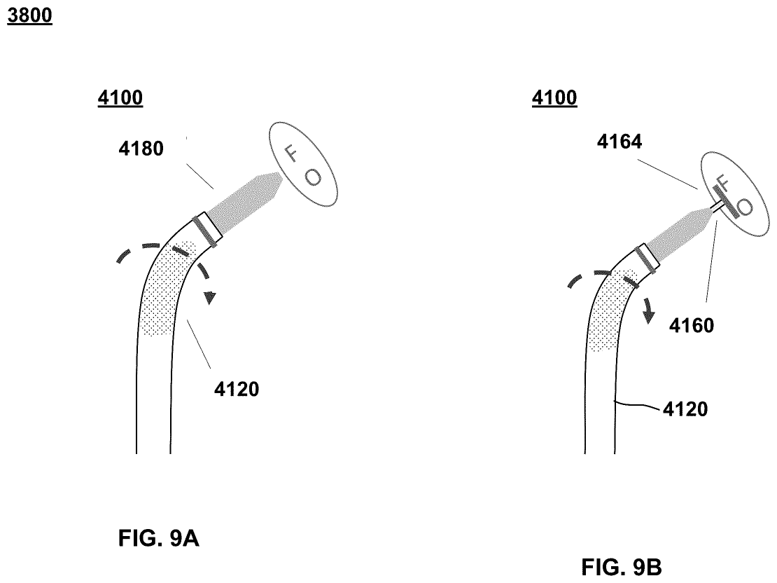

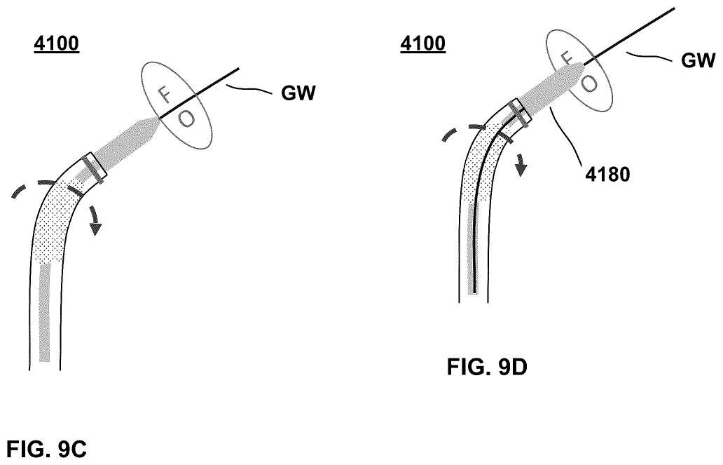

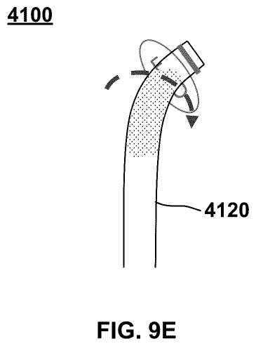

Another embodiment of a delivery device, and a sequence of operation of the device, is illustrated in FIGS. 9A-9F. Delivery device 4100 is similar to devices 3900 and 4000, except that it does not include an inner dilator. It does include a shapeable (e.g., steerable or pre-shaped) catheter 4120, an outer dilator 4180, and an inner guide catheter 4160. In some embodiments, the catheter 4120 may be substantially similar to the catheters 3820, 3920, and/or 4020. As shown in FIG. 9A, device 4100 can be disposed adjacent the fossa FO, and oriented so that the axis of the distal end is pointed towards the fossa. As shown in FIG. 9B, inner guide catheter 4160 can be extended from outer dilator 4180, so that end effector 4164 is deployed, and catheter 4120 can be manipulated, and inner guide catheter extended/retracted, to dispose end effector 4164 on the desired crossing location on the fossa FO. As shown in FIG. 9C, guidewire GW can be extended from inner guide catheter 4160 into contact with fossa FO, energized with RF, and pushed across the fossa into the left atrium LA. As shown in FIG. 9D, inner guide catheter 4160 can be retracted into outer dilator 4180, and dilator 4180 can be advanced along guidewire GW through the puncture in fossa FO. As shown in FIG. 9E, the distal end of catheter 4120 can be extended through fossa FO into left atrium LA, and outer dilator 4180 and inner guide catheter 4160 can be withdrawn from catheter 4120. A therapeutic device can then be delivered through catheter 4120 into left atrium LA.

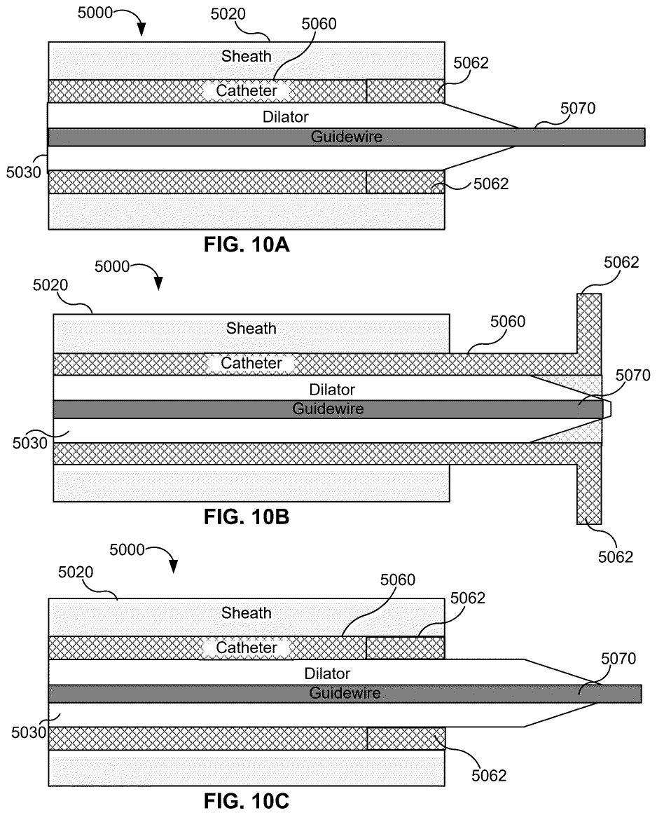

FIGS. 10A-10C are schematic block diagrams of a septum puncture device 5000 (i.e., “the device”) in a navigating configuration, a tenting configuration, and a dilating configuration, respectively. In some embodiments, the septum puncture device 5000 may include a sheath 5020 (e.g., a shapeable sheath, a pre-shaped sheath, a steerable sheath, etc.) defining an inner lumen. In some embodiments, the sheath 5020 may not be steerable. In some embodiments, the sheath 5020 may be semi-rigid and may be provided with a predetermined shape (e.g., a predetermined curve). In some embodiments, the sheath 5020 may be configured such that an operator can manipulate the shape of at least a portion of the sheath 5020 (e.g., via manual adjustment, heat setting, and/or any other suitable method) before the procedure and/or during the procedure. In some embodiments, the sheath 5020 may be shapeable such that the user may remove the sheath 5020 from the body, adjust the shape of the sheath 5020, and then re-insert the sheath 5020 into the body. In some embodiments, the sheath 5020 may be steerable with one or more pull-wires to adjust an angle of at least a portion of the sheath 5020. In some embodiments, the sheath 5020 may be provided in a predetermined non-linear shape suitable for reaching the septum for typical patient population, but can be adjustable prior to and/or during the procedure by the operator to adjust to a particular patient's anatomy.

The septum puncture device 5000 may further include a catheter 5060 slidably disposed in the inner lumen of the sheath 5020 and configured to extend distally relative to the sheath 5020. The catheter 5060 may include an atraumatic end effector 5062 at a distal end thereof and may define an inner lumen in which a dilator 5030 may be slidably disposed. The dilator 5030 may be extendable and retractable through the lumen of the catheter 5060 and may be configured to extend distally relative to the catheter 5060 and the sheath 5020. In some embodiments, the dilator 5030 may define an inner lumen through which a guidewire 5070 (e.g., an RF guidewire) can extend. The guidewire 5070 may be configured to extend distally relative to the dilator 5030. In some embodiments, the guidewire 5070, the dilator 5030, the catheter 5060, and/or the sheath 5020 may be configured to move relative to one another to transition the septum puncture device 5000 between configurations.

In some embodiments, when the septum puncture device 5000 is in the navigating configuration, the guidewire 5070 may extend distally beyond a distal end of the sheath 5020 such that the guidewire 5070 can be navigated toward the heart while the dilator 5030, catheter 5060, and sheath 5020 remain proximal in the body or outside of the body. In some embodiments, the guidewire 5070 may be navigated from the femoral vein, through the IVC, and into the right atrium. Once the guidewire 5070 is disposed in the right atrium, the sheath 5020 including the catheter 5060 and dilator 5030 disposed therein may be advanced along/over the guidewire 5070 into the right atrium. In some embodiments, the guidewire 5070 may have a length such that it can extend beyond the dilator 5030 and form a coiled or pigtailed shape distal to the dilator 5030. In some embodiments, the guidewire 5070 may have a length in a range between about 100 cm and about 300 cm, inclusive of all ranges and subranges therebetween. In some embodiments, the length of the guidewire 5070 may be in a range between about 180 cm and about 230 cm, inclusive of all ranges and subranges therebetween.

Once the septum puncture device 5000 is positioned in the right atrium, a position of the septum (e.g., the fossa) may be located (e.g., via imaging). The septum puncture device 5000 may be transitioned into the tenting configuration in which the catheter 5060 including the end effector 5062 is advanced distally such that the end effector 5062 is deployed (e.g., expands (e.g., self-expands) into an atraumatic shape as it exits and is no longer radially constrained by the sheath). In the tenting configuration, the end effector 5062 can be positioned to contact the fossa and press into the fossa such that the fossa extends into the left atrium. For example, the end effector 5062 may push the fossa into a tent shape that extends into the left atrium. In some embodiments, the end effector 5062 may be echogenic such that the end effector 5062 is visible when ultrasound imaging of the heart is performed. When the device 5000 is not in the tenting configuration (e.g., when the device 5000 is in the navigating configuration and/or the dilating configuration), the catheter 5060 may be disposed in the inner lumen of the sheath 5020 such that the end effector 5062 is biased, and the catheter 5060 and end effector 5062 form a substantially cylindrical shape. In some embodiments, when the device 5000 is in the tenting configuration, the end effector 5062 may be deployed such that it expands into a disc shape (e.g., a donut shape, ring shape, or the like). In some embodiments, the end effector 5062 may tent the septum to (1) locate a target perforation site and/or (2) to pull the tissue taut to increase perforation precision.

In some embodiments, as the septum puncture device 5000 transitions into or is in the tenting configuration, the dilator 5030 may be advanced such that a distal portion of the dilator extends at least half a length of the exposed catheter 5060. In some embodiments, the dilator 5030 may be advanced such that the distal portion of the dilator 5030 extends to a distal end of the inner lumen of the catheter 5060. In some embodiments, the dilator 5030 may be advanced such that the distal portion of the dilator 5030 extends beyond the end effector 5062. In some embodiments, the distal portion of the dilator 5030 may extend beyond the end effector 5062 about 0 mm to about 10 mm, inclusive of all ranges and subranges therebetween. In some embodiments, the distal portion of the dilator 5030 may extend beyond the end effector 5062 about 0.5 mm to about 1 mm, inclusive of all ranges and subranges therebetween. In some embodiments, the dilator 5030 may have a durometer or rigidity that is higher than a durometer of the catheter 5060. Therefore, the dilator 5030 extending through at least a portion of the catheter 5060 may provide additional rigidity and/or stability to the catheter 5060 during tenting of the septum. In some embodiments, extending the dilator 5030 beyond the distal end of the end effector 5062 may allow the distal end of the septum puncture device 5000 to grab (capture, engage) the septum at an oblique angle. In some embodiments, the dilator 5030 may be withdrawn into the catheter 5060 and/or the sheath during tenting. For example, in embodiments in which the sheath 5020 is steerable, the dilator 5030 may be retracted such that the distal end of the dilator 5030 aligns with the distal end of the sheath 5020 during tenting.

In some embodiments, while tenting the septum, the septum may be perforated with the guidewire 5070. In some embodiments, the guidewire 5070 may include an electrode at or near a distal end thereof configured to apply energy (e.g., heat, RF energy, mechanical energy, microwave energy, etc.) to the tissue of the septum to ablate the tissue. In some embodiments, the electrode may be configured to apply RF energy to perforate the target perforation site of the septum. A proximal portion of the guidewire 5070 may be configured to be coupled to an RF generator to delivery RF energy to a distal end portion of the guidewire 5070 and to a target perforation site of a patient. Once energy is applied to the target perforation site and an opening is formed, the guidewire 5070 may be advanced through a target penetration site into the left atrium. The guidewire 5070 may be configured to form a coil as it moves distally out of the dilator 5030 and into the left atrium. In some embodiments, the coil may be atraumatic and prevent the guidewire 5070 from damaging tissue in the heart. The coil of the guidewire 5070 may further prevent the guidewire 5070 from being unintentionally removed from the left and/or right atrium. Once the septum is perforated, the catheter 5060 may be withdrawn proximally. In some embodiments, the catheter 5060 may be withdrawn proximally into the sheath 5020 to transition the end effector away from the deployed configuration. When the catheter 5060 is withdrawn, the dilator may be maintained in a position relative to the perforation site. For example, the dilator 5030 may be cantered on the perforation site.

After perforation, the device 5000 may transition into the dilating configuration, as shown in FIG. 10C. In some embodiments, the dilator 5030 may be advanced distally through the perforation and into the left atrium to dilate the perforation. In some embodiments, the dilator 5030 and the sheath 5020 may be advanced distally together, and a relative position between the dilator 5030 and the sheath 5020 may remain constant while both are advanced. Once the dilator 5030 and the sheath 5020 are disposed in the left atrium, the guidewire 5070, the dilator 5030, and/or the catheter 5060 may be withdrawn proximally out of the sheath 5020 such that just the sheath remains in the left atrium. The sheath 5020 may then be used for the delivery of therapeutics and/or devices. In some embodiments, the device 5000 may enable a “zero exchange procedure” meaning that only one access sheath (the sheath 5020) is used to both cross the septum and for delivering therapy and/or devices.

In some embodiments, the sheath 5020 may be a catheter that is any suitable size for navigating to the atrium and for facilitating delivery of therapeutic devices. For example, the sheath 5020 may be an 8.5 Fr or 12.5 Fr catheter. In some embodiments, the distal end of the sheath 5020 may be more compliant than other portions of the sheath 5020. In some embodiments, the sheath 5020 may include a curved section. In some embodiments, the sheath 5020 may not be steerable and may alternatively include a fixed curved section 5020. For example, the curved section may be shape-set to achieve a predetermined angle. In some embodiments, the sheath 5020 may be steerable and may be configured to achieve the predetermined angle in response to actuation (e.g., of one or more pull wires along a length of the sheath 5020). In some embodiments, the predetermined angle of the sheath 5020 may be in a range of 20 degrees to about 60 degrees, inclusive of all ranges and subranges therebetween. In some embodiments, the predetermined angle of the sheath 5020 may be about 30 degrees to about 50 degrees, inclusive of all ranges and subranges therebetween.

In some embodiments, the dilator 5030 may be deflectable, bendable, shapeable, and/or configured to otherwise accommodate an anatomy of the right atrium such as the fossa angle and/or orientation. In some embodiments, the dilator 5030 may include a hypotube. In some embodiments, the hypotube may be shapeable and/or include a shapeable portion. In some embodiments, the dilator 5030 may be shape-set to an angle corresponding to an anatomy of the right atrium. In some embodiments, the dilator 5030 may include a curved section (e.g., formed by shape-setting) that can achieve a predetermined angle. In some embodiments, the predetermined angle of the curved section of the dilator 5030 may be in a range between about 20 degrees to about 60 degrees, inclusive of all ranges and subranges therebetween. In some embodiments, the predetermined angle of the curved section of the dilator 5030 may be in a range between about 30 degrees to about 50 degrees, inclusive of all ranges and subranges therebetween. In some embodiments, the dilator 5030 may have a total length in a range between about 30 cm to about 150 cm, inclusive of all ranges and subranges therebetween. In some embodiments, the total length of the dilator 5030 may be in a range between about 60 cm to about 90 cm, inclusive of all ranges and subranges therebetween.

In some embodiments, the device 5000 may be configured to interface with an impedance tracking system to track a distal tip of the guidewire. In some embodiments, the distal tip of the guidewire 5070 may alternate between an RF generation impedance monitoring such that the localization of the distal tip can be performed prior to perforation.

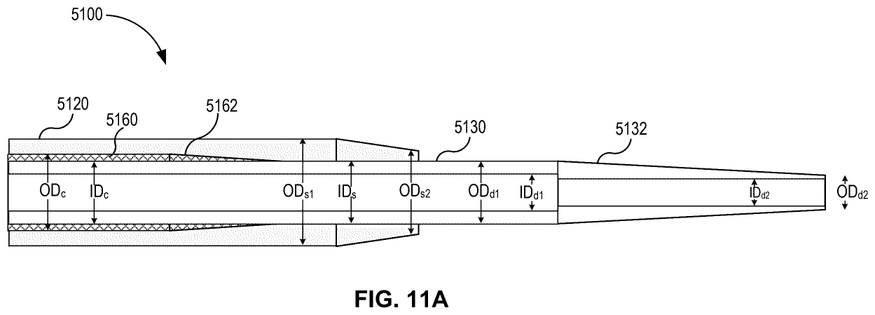

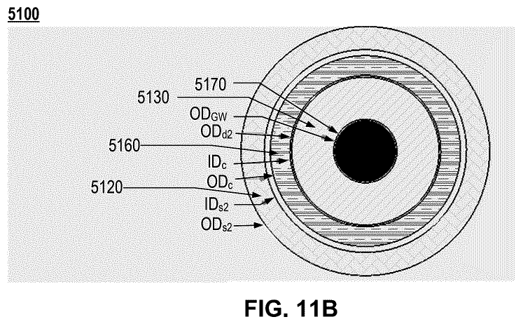

FIG. 11A is a schematic block diagram of a distal end of a septum puncture device 5100 (e.g., “device 5100”) including a sheath 5120, a catheter 5160, and a dilator 5130, according to embodiments. In some embodiments, the septum puncture device 5100 may structurally and/or functionally similar to any of the septum puncture devices described herein, and therefore certain aspects of the septum puncture device may not be described in further detail herein. In some embodiments, the sheath 5120 may have a first outer diameter ODₛ₁ and a second outer diameter ODₛ₂ smaller than the first outer diameter ODₛ₁. In some embodiments, a distal portion of the sheath 5210 may taper from the first outer diameter ODₛ₁ to the second outer diameter ODₛ₂. In some embodiments, the catheter 5160 may include an outer diameter OD_{c} configured to nest inside an inner diameter IDs of the sheath 5120. In some embodiments, when the catheter 5160 is in the delivery configuration, an end effector 5162 of the catheter may be configured to taper inward. In some embodiments, when the catheter 5160 is in the delivery configuration, the end effector 5162 may be configured to form a substantially cylindrical shape with the remainder of the catheter 5160. In some embodiments, when the catheter 5160 is advanced beyond a distal end of the sheath 5120, end effector 5162 may expand to a second outer diameter greater than the first outer diameter OD_{c} (not shown).

The dilator may include a first portion (e.g., a proximal portion) and a second portion 5132 (e.g., a distal portion). In some embodiments, the first portion may not taper, and therefore a first outer diameter Od_{d1} and a first inner dilator ID_{d1} of the dilator 5130 may be constant along the first portion. The second portion 5132 of the dilator may taper from the first outer diameter Od_{d1} to a second diameter Od_{d2} smaller than the first outer diameter Od_{d1}. Additionally, the second portion 5132 of the dilator 5130 may have a second inner diameter ID_{d2} smaller than the first inner diameter. In some embodiments, the dilator 5310 may include a tapered decrease in inner diameter across the second portion 5132. In some embodiments, the dilator may include a stepwise decrease in inner diameter between the first portion and the second portion 5132 of the dilator 5130.

In some embodiments, the sheath 5210 has the inner diameter IDs near a distal end thereof corresponding to a first outer diameter of the dilator OD_{d1}. In some embodiments, the inner diameter IDs of the sheath 5120 may be smaller than the first outer diameter OD_{d1} of the dilator 5130 to promote a smooth transition between the sheath 5120 and the dilator 5130. The sheath may be softer to promote smooth transition, allow dilator to extend therefrom, and to make the distal portion of the sheath more atraumatic, e.g., for when sheath is in left atrium. FIG. 11B is a front cross-sectional view showing the dilator 5130 disposed around the guidewire 5170, the catheter 5100 disposed around the dilator 5130, and the sheath 5120 disposed around the catheter 5160, according to embodiments.

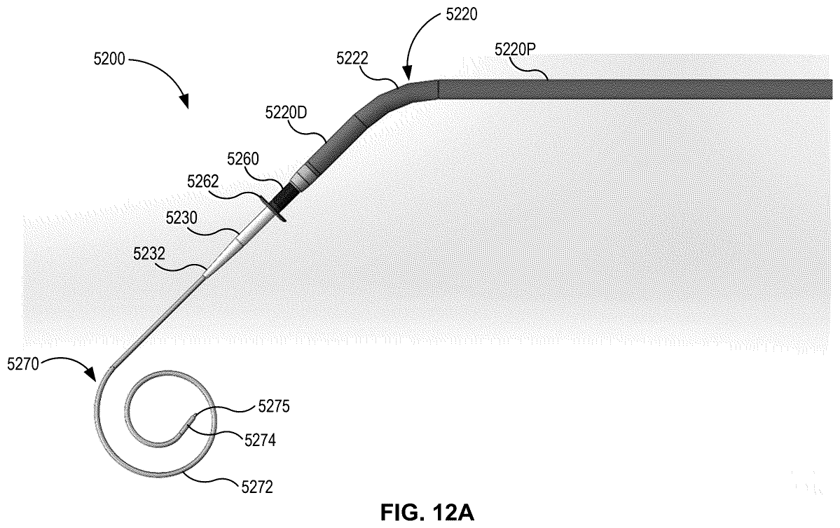

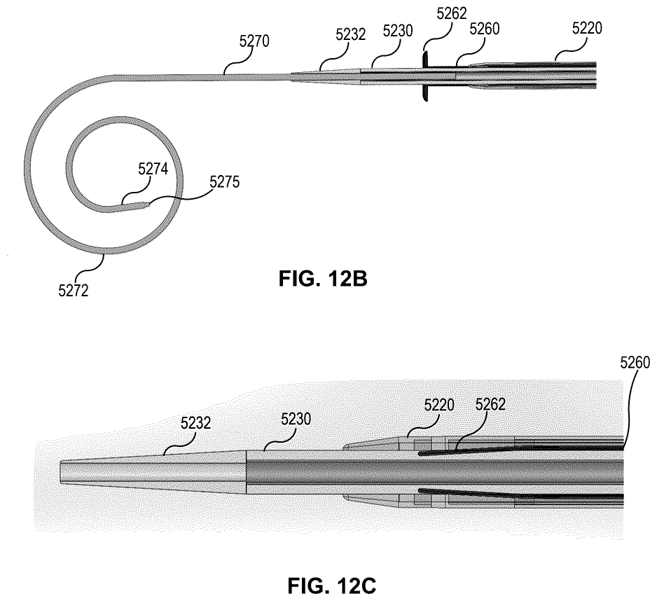

FIG. 12A is an illustration of a distal end of a septum puncture device 5200 including a catheter 5260 slidably disposed in a sheath 5220, a dilator 5230 slidably disposed in the catheter 5260, and a guidewire 5270 slidably disposed in the dilator 5230. As shown, the sheath 5220 includes a proximal portion 5220P and a distal portion 5220D with a curved section 5222 therebetween. In some embodiments, the distal portion 5520D of the sheath 5220 may have a lower durometer than the curved section 5222 and the proximal portion 5220P of the sheath 5220. The sheath 5220 may define an inner lumen through which the catheter 5260 is disposed. The catheter 5260 may include an end effector 5262 at a distal end. As shown, the catheter 5260 may extend distally from the sheath 5220 such that the end effector 5262 is in the deployed configuration. In some embodiments, the catheter 5260 may extend distally from the sheath 5220 such that the end effector 5262 is exposed from the distal end portion of the sheath 5220 to expand from a constrained configuration (e.g., a delivery configuration) in which the end effector 5262 has a first cross-sectional area to an unconstrained configuration (e.g., a deployed configuration) in which the end effector 5262 has a second cross-sectional area greater than the first cross-sectional area. In the deployed configuration, the end effector 5262 is atraumatic.

The catheter 5260 can define an inner lumen through which the dilator 5230 extends. A distal portion 5232 of the dilator 5320 may taper to the distal end such that the dilator 5230 can atraumatically dilate the perforation site. The dilator 5230 defines an inner lumen through which the guidewire 5370 can extend. As shown, the guidewire 5270 when extended distally from the dilator 5230 is configured to transition into a deployed configuration in which the guidewire 5270 may form a curve or coil 5272 (e.g., pigtail, spiral, loop, twist, “J” shape, etc.). In some embodiments, the coil 5272 may be flat or 2-dimensional. In some embodiments, the coil 5272 may include a straight or linear section 5274 at or near a distal end thereof, as shown in FIG. 12A. In some embodiments, the straight section 5274 may have a length such that the straight section 5274 crosses through the septum before the guidewire 5270 begins to form the coil 5272. In some embodiments, the straight section 5274 can be dimensioned to exceed septal thickness such that the guidewire penetrates completely before the coil forms. In some embodiments, the straight section 5274 may have a length in a range of about 0.5 mm to above 3 mm, inclusive of all ranges and subranges therebetween. In some embodiments, a distal tip of the guidewire 5270 may include an electrode 5275 configured to deliver energy to the tissue.

FIG. 12B is a side cross-sectional view of the distal end of the septum puncture device 5200 with the guidewire 5270 in the deployed configuration. FIG. 12C is a close-up, side cross-sectional view of the distal end of the septum puncture device 5200 showing the dilator 5230 extended from a distal end of the catheter 5260 and the sheath 5220 without the guidewire, according to embodiments. As shown, the end effector 5262 of the catheter 5260 is in a delivery configuration such that the catheter 5260 and the end effector 5262 form a substantially cylindrical shape. In some embodiments, the end effector 5262 may taper inwards in the delivery configuration. As shown in FIG. 12C, a distal end of the sheath 5220 may be configured to taper (e.g., inward towards the dilator 5230) such that a transition between the dilator 5230 and the sheath 5220 is smooth (e.g., includes no ridges, sharp transitions, etc.). In some embodiments, the septum puncture device 5200 may be structurally and/or functionally similar to any of the septum puncture devices described herein, and therefore, certain details of the septum puncture device 5200 may not be described herein with respect to FIGS. 12A-12C.

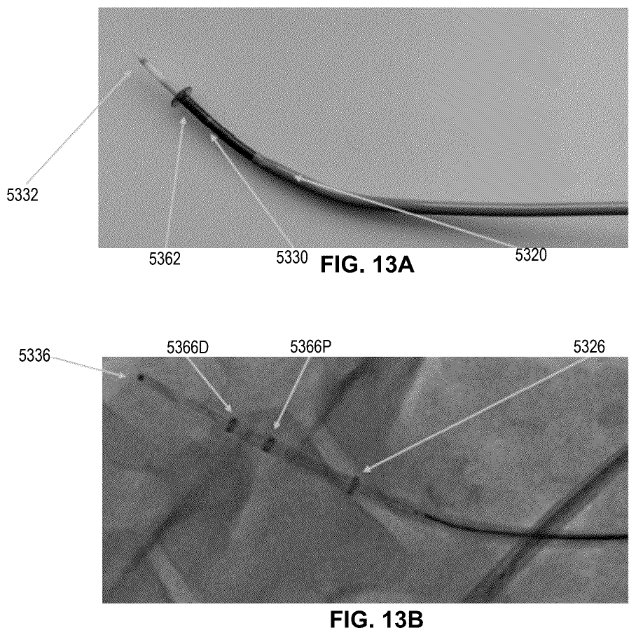

FIG. 13A image of a septum puncture device 5300 showing a position of radiopaque markers, according to embodiments. FIG. 13B shows the radiopaque markers of the septum puncture device when imaged with fluoroscopy, according to embodiments. As shown, the dilator 5332 includes a radiopaque marker 5336 (e.g., a marker band) embedded at or near a distal end thereof. In some embodiments, the radiopaque marker 5336 may be a distance in a range of about 1 mm to about 8 mm from a distal tip of the dilator, inclusive of all ranges and subranges therebetween. The end effector 5362 may have a first radiopaque marker 5336D at a distal end and a second radiopaque marker 5336P at a proximal end thereof. In some embodiments, the first radiopaque marker 5366D at the distal end of the end effector 5362 may be a distance in a range of about 1 mm to about 3 mm from the distal end of the end effector, inclusive of all ranges and subranges therebetween. In some embodiments, the second radiopaque marker 5366P may be a distance in a range of about 6 mm to about 10 mm from the distal end of the end effector 5362, inclusive of all ranges and subranges therebetween. The sheath 5320 may include a radiopaque marker 5326 at a distal end thereof. In some embodiments, the radiopaque marker 5326 of the sheath 5320 may be a distance in a range of about 6 mm to about 10 mm from a distal end of the sheath 5320, inclusive of all ranges and subranges therebetween. Additionally, the guidewire (not shown) may include a radiopaque marker at a distal end thereof. In some embodiments, the markers may include any suitable material such as tungsten, stainless steel, platinum/iridium, etc.

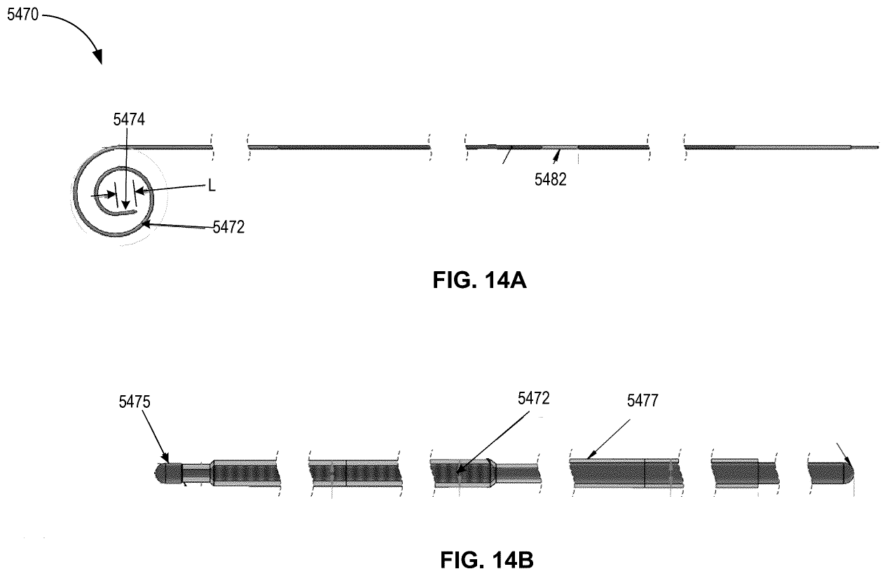

FIGS. 14A-14B show a guidewire 5470 for navigating a septum puncture device including a coil (e.g., pigtail, curled, J-tip, etc.) 5472 at a distal end, according to embodiments. In some embodiments, the coil 5472 includes a number of turns in a range between about 1 turn to about 5 turns, inclusive of all ranges and subranges therebetween. In some embodiments, the coil 5472 may include about 1.5 turns. The length of the coil may be in a range of about 1.5 cm to about 15 cm, inclusive of all ranges and subranges therebetween. In some embodiments, the length of the coil 5472 may be in a range of about 2 cm to about 12 cm, inclusive of all ranges and subranges therebetween. In some embodiments, the pitch of the coil 5472 may be in a range of about 0.1 inches to about 0.5 inches, inclusive of all ranges and subranges therebetween. In some embodiments, the pitch of the coil 5472 may be in a range of about 0.2 inches to about 0.3 inches, inclusive of all ranges and subranges therebetween. In some embodiments, the distal end of the coil 5472 may include a linear section 5474. In some embodiments, the linear section 5474 may have a length L in in a range of about 0.1 cm (1 mm) to about 0.5 cm (5 mm), inclusive of all ranges and subranges therebetween. In some embodiments, the linear section 5474 may have a length L of about 2 mm. In some embodiments, the dilator 5470 may not include any engagement members to control advancement of the guidewire. Instead, a position of the guidewire 5470 relative to the dilator may be determined based on markers disposed along the guidewire 5470 and/or the dilator. In some embodiments, a marker 5482 may be pad printed onto an outside of an insulation jacket 5477 of the guidewire 5470 to indicate when the guidewire is at the distal tip of the device. In some embodiments, the guidewire 5470 may include any suitable material such as a metal, an alloy, a polymer, or a suitable combination thereof. In some embodiments, the guidewire may include, for example, stainless steel and/or fluoropolymer heat shrink. In some embodiments, the guidewire 5470 may include a plurality of layers of material.

In some embodiments, the stiffness of the guidewire 5470 may be determined by the following equation. Flexural Rigitiy=EI Where: E=Elastic (Young's) Modulus and I=Area Moment of Inertia. For a device having a cylindrical cross section:

$$I = \frac{π}{64} ( {D}_{o}^{4} )$$

Where Dₒ=Outside Diameter of Cylinder. In some embodiments, the Modulus of Elasticity may be in a range of about 193 gigapascal (GPa) to about 200 GPa. In some embodiments, the Dₒ of the guidewire may be in a range of about 0.01 inches to about 0.05 inches, inclusive of all ranges therebetween.

In some embodiments, the electrode 5475 disposed on a distal end of the guidewire 5470 may be configured to deliver a predetermined level of energy for a predetermined amount of time. In some embodiments, the predetermined level of energy delivered by the guidewire may be in up to about 50 watts. In some embodiments, the predetermined amount of time may be in a range of about 1 second to about 5 seconds. In some embodiments, the predetermined amount of time may be about 2 seconds.

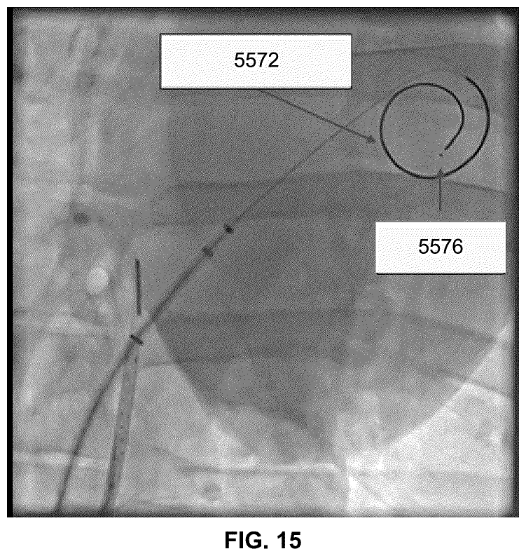

FIG. 15 shows markers of a septum puncture device when imaged with fluoroscopy, according to embodiments. As shown, the guidewire is in the deployed configuration such that the guidewire 5572 forms a coil 5572. As shown, the coil 5572 and a distal tip 5576 of the coil 5572 may include material such that each is visible during imaging.

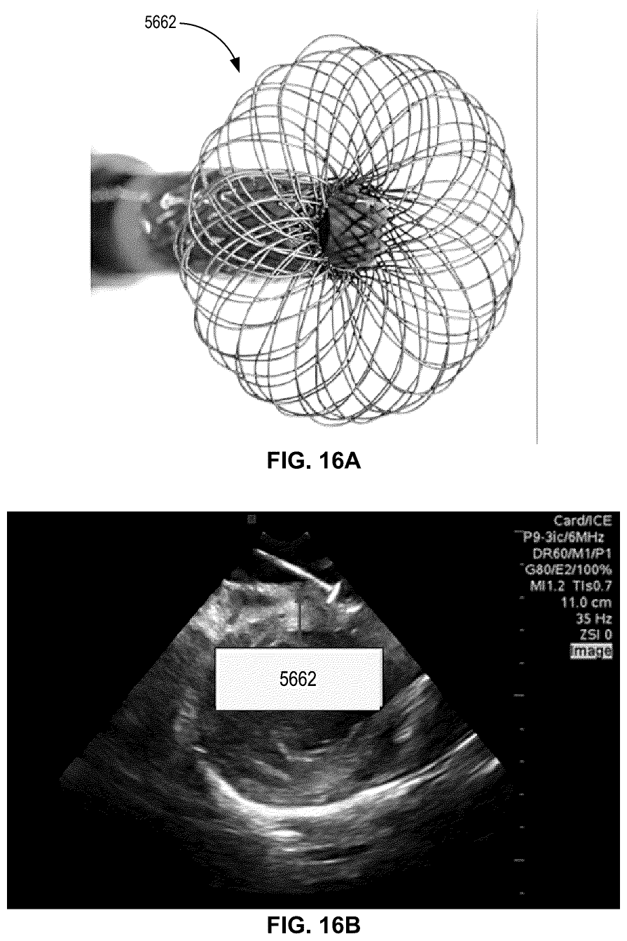

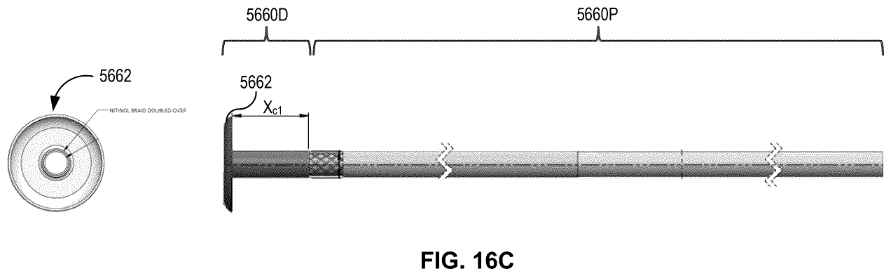

FIG. 16A shows an end effector 5662 of a catheter of a septum puncture device in a deployed configuration, according to embodiments. As shown, the end effector 5662 may be configured to expand (e.g., radially expand) into a donut or disc shape, or any suitable shape that is atraumatic and/or including atraumatic or smooth edges. As shown in FIG. 16B, the end effector 5662 is highly echogenic. In some embodiments, the end effector 5662 may include an echogenic material such that the end effector 5662 may be oriented relative to the septum for tenting. The end effector 5662 may include any suitable material such as a metal or an alloy. In some embodiments, the end effector may include a superelastic material such as braided Nitinol. The braided Nitinol design may scatter soundwaves creating a hyperechoic signal. FIG. 16C shows a diagram of the catheter and the end effector 5662, according to embodiments. In some embodiments, the catheter may include a first portion (e.g., a distal portion) 5660D including the end effector 5662. In some embodiments, the first portion 5660D may include a super elastic material. The catheter may include a second portion (e.g., a proximal portion) 5660P. In some embodiments, the second portion 5660P may include any suitable material such as metal, alloys, polymers, etc. In some embodiments, the second portion 5660P of the catheter may include a polymer (e.g., Pebax®). In some embodiments, a proximal tubular section of the end effector 5662 may be laminated (reflowed) into the standard catheter materials of the first portion 5660D of the catheter to secure the exposed disc portion to the catheter.

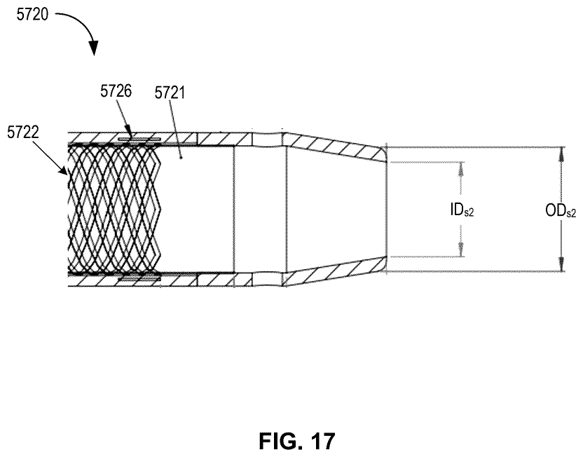

FIG. 17 shows a diagram of a sheath 5720 of a septum puncture device configured to have a catheter, dilator, and guidewire extended therethrough, according to embodiments. The sheath 5720 includes an inner lumen 5721 configured to receive the catheter. As shown, a distal end of the sheath 5720 tapers to transition to a diameter of the dilator disposed therein. As shown, the distal end of the sheath 5720 has an inner diameter in a range of about 0.08 in to about 0.1 in, inclusive of all ranges and subranges therebetween. In some embodiments, the distal end of the sheath 5720 has an outer diameter in a range of about 0.1 in to about 0.11 in, inclusive of all ranges and subranges therebetween. In some embodiments, the sheath 5720 includes a curved section 5722. In some embodiments, the curved section 5722 may include a laser cut super elastic material (e.g., Nitinol hypotube). The sheath 5720 may further include a marker band 5726 arranged proximal to a distal portion of the sheath 5720 and configured to help the operator localize the sheath 5720 during the procedure.

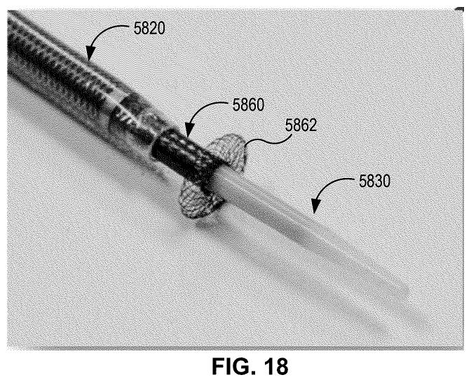

FIG. 18 shows a distal end of the septum puncture device, according to embodiments. As shown, the sheath 5820 is configured to taper at a distal end such that a transition between the sheath 5820 and a catheter 5860 disposed therein is smooth. In some embodiments, the distal end of the sheath 5820 may conform to a diameter of the catheter 5860. The catheter 5860 can extend distally from the sheath 6820 and may include an end effector (e.g., a disc shape end effector) 5862. The dilator 6830 may extend distally from the catheter 5860 and may taper at a distal end thereof.

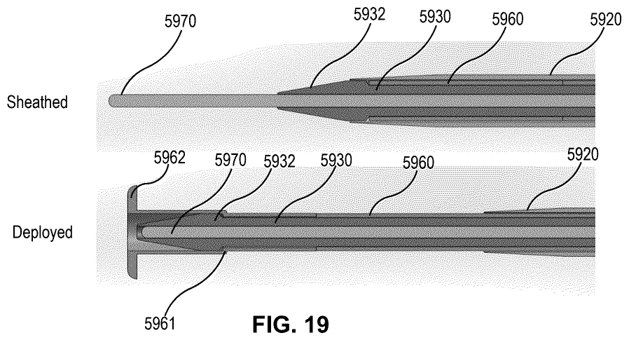

FIG. 19 shows a catheter 5960 of a septum puncture device in a sheathed position and a deployed position, according to embodiments. The septum puncture device may include a sheath 5920, a catheter 5960 slidably disposed in the sheath 5920, a dilator 5930 slidably disposed in the catheter 5960, and a guidewire 5970 slidably disposed in the dilator 5930. As shown, when the septum puncture device is in the sheathed position, a distal portion 5932 of the dilator 5930 may extend beyond a distal end of the catheter 5960 while still being partially sheathed by the sheath 5920. In some embodiments, the distal portion 5932 of the dilator 5930 may be configured to extend beyond the distal end of the sheath 5920. In some embodiments, an outer diameter of the dilator 5932 may increase from the distal tip until the outer diameter reaches a maximum diameter, then may decrease (e.g., may decrease step-wise or gradually). For example, the outer diameter of the dilator 5932 may include a ridge, a detent, an enlarged section. In some embodiments, the ridge of the dilator 5932 may be configured to abut a distal end of the catheter 5930 when in the sheathed position to hold the dilator 5930 in position relative to the catheter 5960 and the sheath 5920. For example, a proximal end of the distal portion 5932 of the dilator 5930 may be configured to abut an outer diameter of the catheter tip to create a smooth transition from the sheath 5920 to the dilator 5930. In some embodiments, a section of the end effector 5962 may be unsupported/unlaminated so that the catheter 5960 can accommodate the maximum diameter of the dilator 5930 when the catheter 5960 is advanced distally over the dilator 5930. In some embodiments, the catheter 5960 may include a braided Nitinol sheath configured to expand when in the deployed configuration to a larger diameter such that the braided Nitinol sheath may be advanced beyond the dilator 5930. In some embodiments, the catheter 5960 in the deployed configuration may include a proximal portion having a first diameter and a distal portion having a second diameter greater than the first diameter. In some embodiments, the first portion and the second portion of the catheter 5960 may both have the second diameter when in the sheathed or delivery configuration. In some embodiments, a transition from the first diameter to the second diameter may form an engagement portion 5961 (e.g., a surface feature, an indentation, a bump, a detent, etc.) configured to engage the ridge of the dilator 5930 when the catheter 5960 is in the deployed configuration. In some embodiments, the engagement portion 5961 may be positioned a predetermined distance from the end effector 5962 such that the dilator 5930 is held in place relative to the end effector 5962. In some embodiments, the engagement portion 5961 may hold the dilator 5930 such that the dilator 5930 extends along at least half the length of the catheter 5960 to provide stability and/or rigidity.

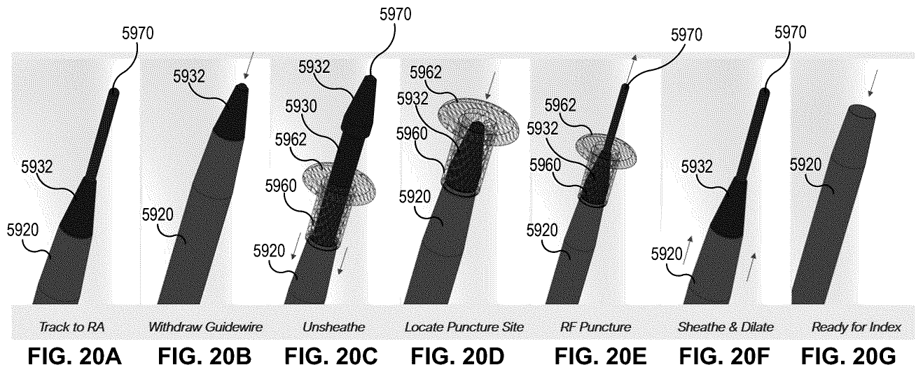

FIGS. 20A-20G show different configurations of the septum puncture device of FIG. 19 during a procedure of puncturing a septum of a heart, according to embodiments. As shown in FIG. 20A, the device is in a navigation in which the guidewire 5970 extends distally beyond the distal portion 5932 of the dilator 5930. In some embodiments, the distal portion 5932 of the dilator 5930 may extend beyond a distal end of the sheath 5920. The device in the navigation configuration may be navigated into the right atrium of the heart.

FIG. 20B shows the septum puncture device after the dilator 5930 and the sheath have been tracked over the guidewire 5970 and disposed in the right atrium of the heart. As shown, the guidewire 5970 is withdrawn proximally into the dilator 5930. Once the dilator 5930 and the sheath 5920 are disposed in the right atrium, the catheter 5960 may be unsheathed, as shown in FIG. 20C-20D. As shown, the dilator 5930 may be advanced distally beyond the sheath 5920 so that the catheter 5960 may be allowed to advance distally. For example, advancing the dilator 5930 distally may disengage the ridge of the dilator 5930 from the catheter 5960 such that catheter 5960 may be advanced. Once the catheter 5960 is advanced a predetermined distance from the distal end of the sheath 5920, the dilator 5930 may then be withdrawn proximally to a predetermined position relative to the catheter 5960, thereby transitioning the device into the tenting configuration. For example, as shown in FIG. 20D, the dilator 5930 may be withdrawn until a distal tip of the dilator 5930 aligns with a distal tip of the end effector 5962. In the tenting configuration, the device may be navigated to locate the puncture site. Once the puncture site is located, and the septum is tented, the device may transition into the perforating configuration, as shown in FIG. 20E.

In the perforating configuration, the guidewire 5970 may be advanced distally from the dilator 5930 and configured to contact the septum. In some embodiments, RF energy may be applied to the tissue via the guidewire 5970 to perforate the tissue. Once the tissue is perforated, the device may transition to the dilating configuration, as shown in FIG. 20F. In the dilating configuration, the end effector 5962 may be withdrawn proximally into the sheath 5920. In some embodiments, the dilator 5930 may be configured to continue contacting the septum when the end effector 5962 is withdrawn. Once the end effector 5962 is withdrawn, the dilator 5930 may be advanced distally through the perforation site and into the left atrium to dilate the perforation site. The sheath 5920 may be maintained at a fixed position relative to the dilator 5930 and may be advanced through the dilated perforation site and into the left atrium. Once a distal portion of the sheath 5920 is disposed in the left atrium, the guidewire 5970, catheter 5960, and dilator 5930 may be retracted proximally leaving the sheath in the left atrium, as shown in FIG. 20G. Now the sheath is ready to be used to delivery other devices. The septum puncture device may be structurally and/or functionally similar to any of the septum puncture devices described herein, and therefore certain aspects of the septum puncture device may not be described herein with respect to FIGS. 19, 20A-20G.

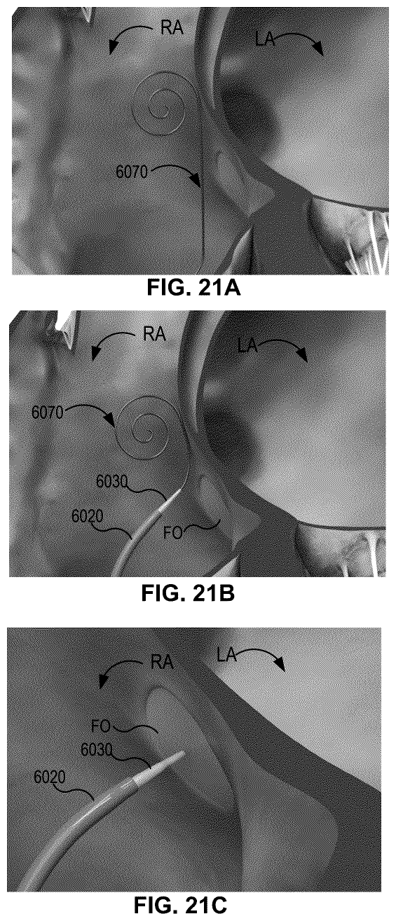

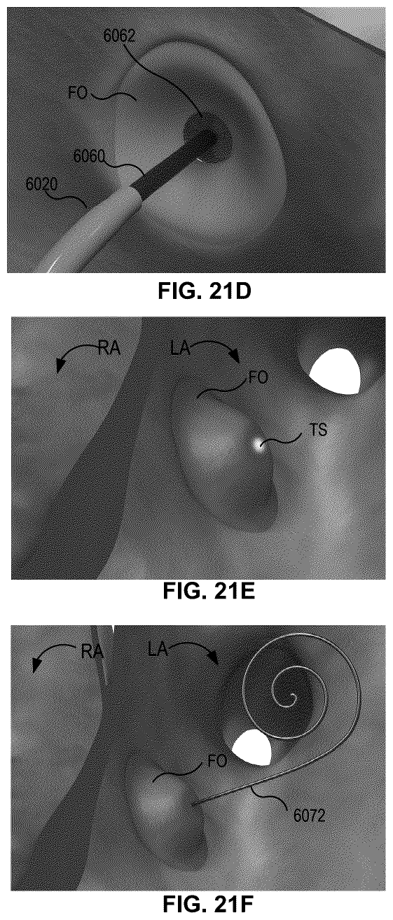

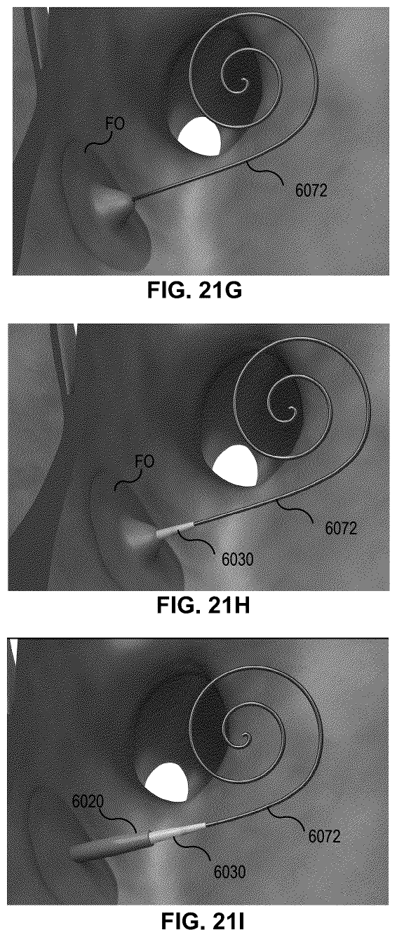

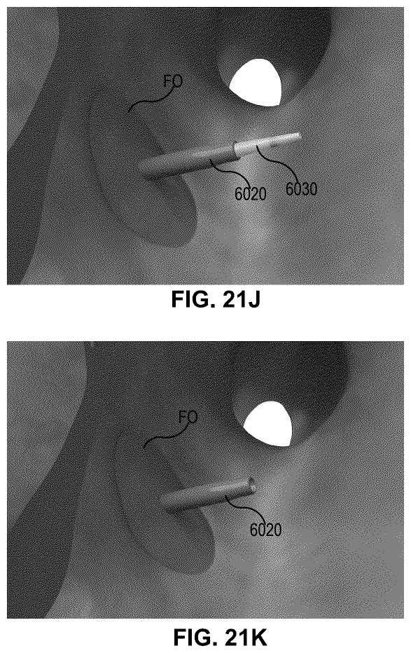

FIGS. 21A-21K show a method for puncturing a fossa FO of a heart using the septum puncture device of FIGS. 12A-12C, according to embodiments. FIG. 21A shows a guidewire 6070 advanced into the right atrium RA. As shown, the guidewire 6070 is in the deployed configuration (e.g., the atraumatic configuration) such that the guidewire 6070 forms a coil. With the distal end portion of the guidewire 6070 disposed within the right atrium RA of the heart of the patient, the sheath 6020, catheter (not shown), and dilator 6030 can be advanced over the guidewire 6070 and to dispose a distal end portion of the sheath 6020 and a distal end portion of the dilator 6030 into the right atrium RA, as shown in FIG. 21B. FIG. 21C shows the distal end of the sheath 6020 with the dilator 6030 extending therefrom being positioned near the septum. In some embodiments the distal end of the guidewire and optionally portions of the septum puncture device can first be advanced from the IVC and into the SVC, and then withdrawn into the right atrium (similar to as shown in FIG. 21B), whereas in some embodiments the distal end of the guidewire and the septum puncture device can be advanced from the IVC to the right atrium without entering the SVC.

FIG. 21D shows advancing the catheter 6060 distally from the sheath 6020 and towards a fossa FO of the heart. As shown, the catheter 6060 has an atraumatic end effector 6062 extending from its distal end. The advancing the catheter 6060 includes tenting the target perforation site of the fossa FO with the end effector 6062 (e.g. such that a portion of the fossa FO extends into the left atrium LA), as shown in FIG. 21D. In some embodiments, the end effector 6062 may be visualized using ultrasound from outside the patient during the tenting. In some embodiments, the end effector 6062 may be visualized via at least one of transthoracic echocardiography, transesophageal echocardiography, or intracardiac echocardiography. In some embodiments, during the tenting, (1) the distal end portion of the catheter 6060 may extend a distance from the distal end of the sheath 6020 and (2) the distal end portion of the dilator 6030 may extend a predetermined length through the lumen of the catheter 6060 distal to the distal end of the sheath 6020. For example, the dilator 6030 may extend through an entirety of the exposed catheter 6060. In some embodiments, the dilator 6030 may extend half the length of exposed catheter 6060 (e.g., the catheter distal to the distal end portion of the sheath 6020). In some embodiments, before or during the tenting, the dilator 6030 may be advanced beyond the end of the end effector 6062 and into contact with the fossa FO. As shown in FIG. 21E, after the end effector 6062 tents the fossa FO, a distal end of the guidewire 6070 including an electrode may be configured to contact the target perforation zone and perforate the perforation zone. After the perforating, the guidewire 6070 may then be advanced into the left atrium LA, as shown in FIG. 21F. As shown, when the guidewire 6070 is advanced, the guidewire 6070 is allowed to coil into a pigtail 6072 as the distal end portion of the guidewire 6070 exits a lumen of the dilator 6030 within the left atrium LA. In some embodiments, the pigtail 6072 is atraumatic and prevents the guidewire 6070 from accidentally being withdrawn out of the left atrium LA and/or right atrium RA. In some embodiments, when the guidewire 6070 coils, the distal end of the guidewire 6070 may include a linear section.

After perforating and before dilating, the catheter 6060 may be withdrawn into the sheath 6020 such that the end effector 6062 is out of contact with the fossa FO. In some embodiments, after the perforating and with the distal end portion of the guidewire 6070 disposed within the left atrium LA, the catheter 6060 may be withdrawn proximally within the sheath 6062 while maintaining the portion of the fossa FO in the left atrium LA by pushing on the portion of the fossa FO with a distal end of the dilator 6030. As shown in FIG. 21G, when the end effector 6062 is withdrawn proximally, the fossa FO may fold around the dilator 6030. With the distal end portion of the guidewire 6070 disposed within the left atrium LA and with the end effector 6062 withdrawn, the perforated target perforation site may be dilated by advancing the distal end portion of the dilator 6030 and the sheath 6020 along the guidewire 6070 and into the left atrium LA, as shown in FIG. 21H-21I. In some embodiments, the dilator 6030 and the sheath 6020 may be fixed relative to one another as the dilator 6030 and the sheath 6020 are advanced through the fossa FO and/or across the septum. After the dilating, the distal end portion of the guidewire 6070 may be withdrawn proximally into the dilator 6030, as shown in FIG. 21J. Then, the dilator 6030 may be withdrawn proximally within the sheath 6020 and from the left atrium, leaving the distal end portion of the sheath 6020 disposed within the left atrium, as shown in FIG. 21K.

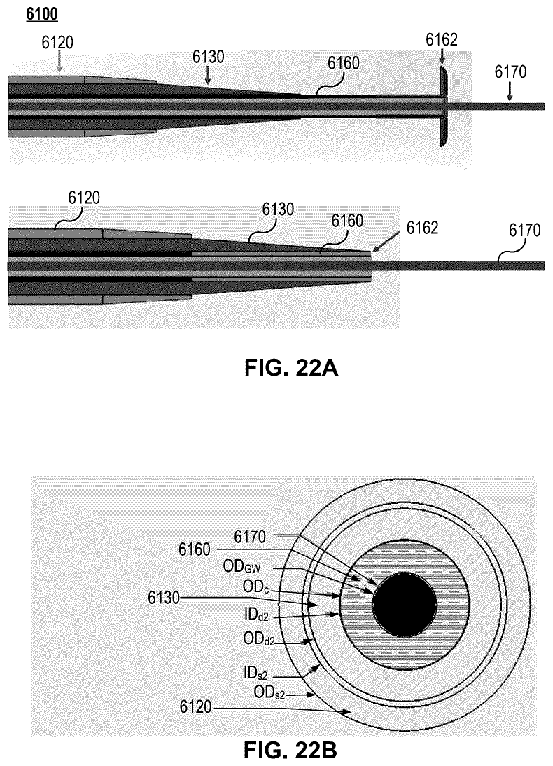

FIG. 22A shows a cross-sectional side view of a septum puncture device 6100 including an outer dilator 6130 disposed inside a sheath 6120 and around a catheter 6160, according to embodiments. As shown, the dilator 6130 defines an inner lumen through which the catheter 6160 may be slidably disposed. The catheter 6160 defines an inner lumen through which the guidewire 6170 may be slidably disposed. In some embodiments, during tenting, the dilator 6130 may extend at least partially along a length of the exposed catheter 6160. In some embodiments, the dilator 6130 may extend to the distal end of the end effector 6162 during tenting to provide rigidity. In some embodiments, during dilating, the catheter 6160 may be withdrawn inside the dilator 6130 such that the dilator 6130 can be disposed through the perforated target site.

FIG. 22B shows a cross-sectional front view of the septum puncture 6100 device of FIG. 22A, according to embodiments. As shown, the catheter 6160 is disposed around the guidewire 6170, the outer dilator 6130 is disposed around the catheter 6160, and the sheath 6120 disposed around the outer dilator 6130, according to embodiments.

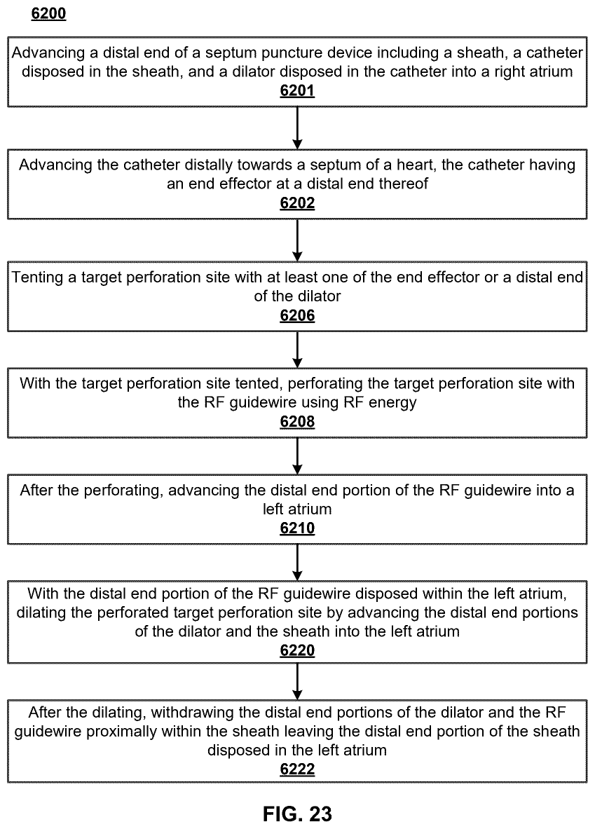

FIG. 23 is a flow chart diagram of an example method 6200 of puncture a septum of a heart of a patient using a septum puncture device (e.g., any of the septum puncture devices described herein, according to embodiments. The method 6200 may be similar to the method described in FIGS. 21A-21K, and therefore certain details of the method may not be described herein with respect to FIG. 23.

As shown, the method 6200 includes with a distal end of a guidewire disposed in the right atrium, advancing a distal end of a septum puncture device including a sheath, a catheter disposed in the sheath, and a dilator disposed in the catheter into a right atrium, at 6201. At 6202, the method includes advancing the catheter distally towards a septum of a heart, the catheter having an end effector at a distal end thereof. The method may include tenting a target perforation site with at least one of the end effector or a distal end of the dilator, at 6206. At 6208, with the target perforation site tented, perforating the target perforation site with the guidewire (e.g., using RF energy, cutting the tissue, etc.). In some embodiments, after the perforating, the method may include withdrawing the guide catheter proximally such that the end effector is sheathed. In some embodiments, a distal end of the dilator may be kept in contact with and/or push the septum such that a portion of the septum remains in the left atrium. After the perforating, the method may include advancing the distal end portion of the guidewire into the left atrium through the perforation site, at 6220. At 6222, after the dilating, the distal end portion of the dilator and the guidewire may be withdrawn proximally within the sheath, thereby leaving the distal end of the sheath disposed in the left atrium. Therefore, the sheath may be used for the delivery of therapies and/or therapeutic devices into the left atrium.

Although various embodiments described herein focus on using a puncture device to puncture a septum of a heart, the functionality provided by various puncture devices described herein can be desirable in other procedures and in other parts of a patient. For example, many procedures exist in which it would be desirable to be able to provide a stable, precise, safe, and repeatable lateral puncture. In some instances, for example, any of the puncture devices described herein could be used to facilitate a tricuspid annuloplasty. The puncture device, for example, could be arrange such that a central axis of its main shaft is parallel to a plane of the tricuspid valve, and so the puncture device could provide lateral or perpendicular access to the annulus of the tricuspid, e.g., to deliver sutures, screws, or other anchoring devices for purposes of a tricuspid annuloplasty.

As another example, the puncture devices described herein could provide an access and a direct vector to a coronary sinus of a heart, to, e.g., insert or deliver a wire, a catheter, a mitral valve repair device, pacemaker leads, etc. into the coronary sinus.

As another example, the puncture devices described herein could be used for delivering therapeutic repair or replacement devices to a mitral valve within a heart. If, for example, a side catheter guide or a side catheter disclosed herein were extended further, and beyond about 90 degrees, the side catheter could be directed into the LA and towards the mitral valve. In some instances, the natural trajectory of the side catheter in some of the embodiments described herein would be angled or directed towards the mitral valve if extended or advanced a suitable distance. For example, as the side catheter assumes its laterally deflected shape or orientation, it may be curved or possess an arc, such that further advancement relative to the main shaft results in the side catheter advancing along such a curvature or arc such that the distal end of the side catheter turns or is further laterally deflected towards the mitral valve. Said another way, in some instances, advancement of the side catheter from its delivery configuration to an advanced/deployed configuration can include the distal end of the side catheter being laterally deflected up to about 180 degrees.

As another example, the puncture devices described herein could incorporate an intracardiac echo catheter to enable accelerate transseptal puncture.

As another example, the puncture devices described herein could be used in connection with cardiac arrest. In such instances, for example, one or more puncture devices could be used in combination with a broad, curved catheter, to enable a guidewire to be directed or delivered from the femoral vein, across the FO, through the mitral valve and out the left ventricular outflow tract (“LVOT”)/aortic valve. In some embodiments a balloon/flow-directed catheter would be advanced across the FO, into the LA, across the mitral valve and then across the LVOT/aortic valve; the balloon, for example, would serve to “flow direct” the catheter out the LVOT and across the aortic valve into the aorta. Once in position, the wire could be used as a track for a small catheter that could provide extracorporeal membrane oxygenation (“ECMO”) and oxygen to the brain. A distal end of the catheter in the aorta would be the outflow, and more proximal ports (e.g., in the RA or the IVC) would be the inflow to the pump.

As another example, the puncture devices described herein could be used in an aorta to facilitate delivery of branch vessel stents, to deliver coils to branch vessels, or to deliver a screen for cerebral embolic protection to the head vessel.

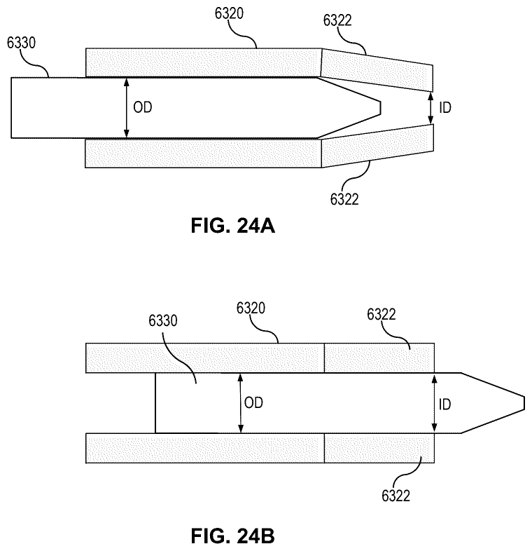

FIGS. 24A-24B show schematic block diagrams of a dilator 6330 and sheath 6320 of a septum puncture device. FIG. 24A shows the dilator 6330 disposed proximally within the shaft 6320. FIG. 24B is a schematic block diagram of the septum puncture device with the dilator 6330 advanced distally from the lumen of the shaft 6320. In some embodiments, an inner diameter ID of the sheath 6320 can be less than a maximum outer diameter OD of the dilator 6330 when a portion of the dilator 6330 having the maximum outer diameter is disposed proximal to the distal end portion of the sheath 6320 and/or distal to (e.g., if the dilator tapers both distally and proximally as shown in FIGS. 19-20G) the distal end portion of the sheath 6320. Therefore, when the distal portion 6322 of the sheath 6320 is unconstrained by the dilator 6330, the inner diameter of the distal portion 6322 of the sheath 6320 is less than the maximum outer diameter OD of the dilator 6330. In some embodiments, the sheath 6320 can include a distal portion 6322 having a lower durometer than a portion proximal to the distal portion 6322. Therefore, the distal portion 6322 having the lower durometer can be configured to expand and/or contract radially and/or laterally to accommodate the maximum outer diameter OD of the dilator 6330 as the dilator 6330 is advanced distally through the sheath 6320, as shown in FIG. 24B, or withdrawn proximally into the sheath 6320. In some embodiments, the distal end portion 6322 of the sheath 6320 having the lower durometer can be configured to make contact with or form a seal with an outer surface of the dilator 6330 as a portion of the dilator 6330 is advanced distal to the sheath 6320. Therefore, the sheath 6320 may form sufficiently tight contact with the dilator 6330 to prevent the sheath 6320 from separating from the outer surface of the dilator 6330 and/or from peeling away from the dilator 6330.

In some embodiments, the distal portion 6322 of the sheath 6320 can be formed of a material that is different than the portion of the sheath 6320 proximal to the distal portion 6322. The material can have higher flexibility or pliability such that the distal portion 6322 can expand and/or contract when the dilator 6330 and/or catheter are disposed therethrough. In some embodiments, a durometer of the distal portion 6322 of the sheath 6320 can be between about shore 00 through shore 55D, inclusive of all values and subranges therebetween. In some embodiments, the durometer of the distal portion 6322 of the sheath 6320 can be between about shore 20D to about shore 55D, inclusive of all values and subranges therebetween. In some embodiments, the distal portion 6322 of the sheath 6330 can be formed from any suitable material that provides i) sufficient elongation at break to dilate from a minimum inner diameter to a maximum inner diameter, ii) sufficient tear strength, iii) a low compression set, iv) ethylene oxide (EtO) sterilization compatible, and/or v) suitability for thermally bonding with the material of the catheter shaft. In some embodiments, the distal portion 6322 of the sheath 6330 can be formed from a material such as, for example, medical-grade liquid silicone rubber (LSR), High-consistency rubber (HCR) silicone, Thermoplastic polyurethane (TPU) (e.g., Tecoflex, Pellethane, Carbothane), low-durometer Pebax (e.g., 25D, 35D, 40D), Polyether-polyamide copolymers, Styrenic block copolymers (e.g., Kraton, Medalist), Medical-grade thermoplastic elastomers (TPE), SEBS-based elastomers, Polyolefin elastomers (e.g., Engage, Exact plastomers), synthetic polyisoprene, blended polymer systems (e.g., Pebax/TPU blends thermoplastic vulcanizate (TPV), nitrile, ethylene propylene diene monomer (EPDM), or butyl rubber. In some embodiments, the distal portion 6322 of the sheath 6330 can be formed from a material including a predetermined ratio of polymers.

In some embodiments, a minimum inner diameter ID of the distal portion 6322 of the sheath 6320 (e.g., measured at the distal tip as shown in FIG. 24A) can be in a range between about 0.075 in and about 0.095 in (or 1.905 mm to about 2.413 mm), inclusive of all values and subranges therebetween. In some embodiments, the inner diameter ID of the distal portion 6322 of the sheath can reach a maximum inner diameter (e.g., when expanded by the dilator 6330 and/or the catheter) of about 0.110 in to about 0.130 in (or 2.794 mm to about 3.302 mm), inclusive of all values and subranges therebetween. In some embodiments, the inner diameter ID and/or the outer diameter of the distal portion 6322 of the sheath 6320 can be configured to increase or expand by about 0.030 in to about 0.050 in (or 0.762 mm to about 1.27 mm), inclusive of all values and subranges therebetween. In some embodiments, the inner diameter ID and/or the outer diameter of the distal portion of the sheath 632 can be configured to increase or expand by about 30% to about 50%, inclusive of all values and subranges therebetween. In some embodiments, the inner diameter ID and/or the outer diameter of the distal portion of the sheath 632 can be configured to increase or expand by about 40% to about 45%, inclusive of all values and subranges therebetween. In some embodiments, the distal end portion 6322 of the sheath 6320 may be configured to contract radially and/or laterally to form a gradual or smooth transition between the outer surface of the dilator 6330 and the sheath 6320.

In some embodiments, the outer diameter of the sheath 6320 may taper from a first outer diameter to a second outer diameter (e.g., at a distal tip of the sheath 6320) smaller than the first diameter. In some embodiments, the second outer diameter the sheath 6320 can be in a range between about 0.090 in to about 0.105 in (or 2.286 mm to about 2.667 mm), inclusive of all values and subranges therebetween. In some embodiments, the second outer diameter of the sheath 6320 may be no greater than about 0.10 mm to about 0.65 mm than the maximum outer diameter OD of the dilator 6330. In some embodiments, the second outer diameter of the sheath 6320 may be no greater than about 0.1 mm to about 0.25 mm. In some embodiments, the distal portion 6322 can taper from the first diameter to the second diameter over a distance of about 3 mm to about 10 mm, inclusive of all values and subranges therebetween. In some embodiments, a slope of the taper measured as a change in radius of the sheath 6320 over a distance from the distal tip of the sheath 6320 may be no greater than about 0.12 such that the sheath 6320 forms a smooth transition from the outer surface of the dilator 6330. In some embodiments, an average slope of the taper may be about 0.06 to about 0.12, inclusive of all values and subranges therebetween. In some embodiments, the outer diameter OD of the dilator 6330 can be similar or the same in form or function to the outer diameter OD_{d1} described in FIGS. 11A-11B. In some embodiments, the inner diameter ID of the sheath 6320 can be similar or the same in form or function to the inner diameter IDs described in FIGS. 11A-11B.

In some embodiments, the sheath 6330 can include a lubricant or coating disposed thereon (e.g., coating at least a portion of an inner and/or outer surface thereof). In some embodiments, the lubricant can reduce drag or friction between the sheath 6320 (e.g., the distal portion 6322 of the sheath 6320) and the shaft or dilator 6330 passing through the sheath 6320. In some embodiments, the lubricant can be disposed on an inner surface of the distal portion 6322 of the sheath 6320. Alternatively or additionally, the lubricant can be disposed on an outer surface of a distal portion of the shaft (e.g., the catheter shaft including the end effector). In some embodiments, the lubricant can be applied the sheath 6320 and/or the shaft from the distal tip to about 5 cm to about 15 cm proximal to the distal tip. In some embodiments, the lubricant can be applied to the surface of the sheath and/or catheter shaft. In some embodiments, the lubricant can be adhered or bonded to the surface of the sheath and/or catheter shaft. Reducing drag or friction between the sheath 6320 and devices disposed therethrough can prevent buckling of or disbanding (e.g., particle formation) of the sheath 6320 (e.g., during crossing of the septum and/or advancement/withdrawal of devices through the sheath 6320). In some embodiments, the lubricant can have a viscosity in a range between about 1,000 cP and about 12,500 cP, inclusive of all values and subranges therebetween.

For clarity, FIGS. 24A-24B only show the dilator 6330 and sheath 6320 of the septum puncture device; however, it should be appreciated that the septum puncture device in FIGS. 24A-24B can include a catheter slidably disposed through a lumen of the sheath 6320 around the dilator 6330 and/or a guidewire slidably disposed through a lumen of the dilator 6330 (e.g., as shown in FIGS. 10-11). In some embodiments, the catheter can be configured to extend distally relative to the sheath 6320. The catheter can include an end effector disposed at a distal end thereof. In some embodiments, the end effector can be configured to transition between a first configuration in which the end effector has a first diameter to a second configuration in which the end effector has a second diameter greater than the first diameter. In some embodiments, the end effector forms an atraumatic shape in the second configuration. In some embodiments, the end effector can be configured to automatically transition from the first configuration to the second configuration as the end effector is advanced distal to the sheath 6320. In some embodiments, when the end effector is in the first configuration, a portion of the end effector extends distally from the catheter (e.g., such that the catheter forms a single layer of material). In some embodiments, a distal end of the end effector is configured to move proximally when the end effector transitions from the first configuration to the second configuration. The septum puncture device can be structurally and/or functionally similar to the septum puncture devices shown herein, and therefore, the septum puncture device is not described herein with respect to FIGS. 24A-24B.

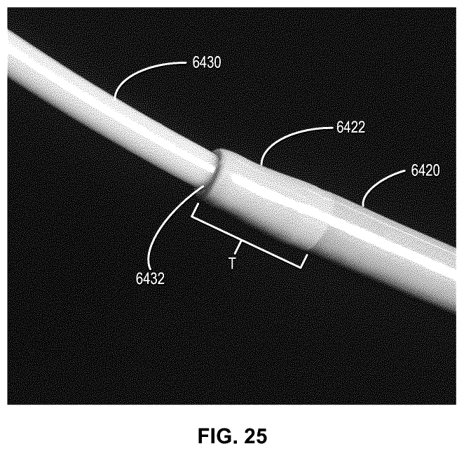

FIG. 25 is an image depicting a distal portion of a septum puncture device, according to embodiments. As shown, the septum puncture device can include a sheath 6420 and a dilator 6430 slidably disposed within the sheath 6420 (e.g., through a lumen of a catheter within the sheath 6420). The sheath 6420 can include a distal end portion 6422 having lower durometer than a proximal portion of the sheath 6420. The distal end portion 6422 of the sheath 6420 can include a tapered portion T configured to taper from a first diameter at a proximal end to a second diameter smaller than the first diameter at a distal end. In some embodiments, an inner diameter along at least a region of the distal end portion 6422 can be smaller than an outer diameter of a portion of the dilator 6430 (e.g., a maximum outer diameter of the dilator 6430). As shown, the distal end portion 6422 can be formed from a different material than the proximal portion of the sheath. The material of the distal end portion 6422 can have a greater pliability (e.g., lower durometer) and can be configured to form sufficient contact with (e.g., form a seal with) an outer surface of the dilator 6430. The septum puncture device can be structurally and/or functionally similar to the septum puncture device described in FIGS. 24A-24B, and therefore, the septum puncture device is not described herein with respect to FIG. 25.

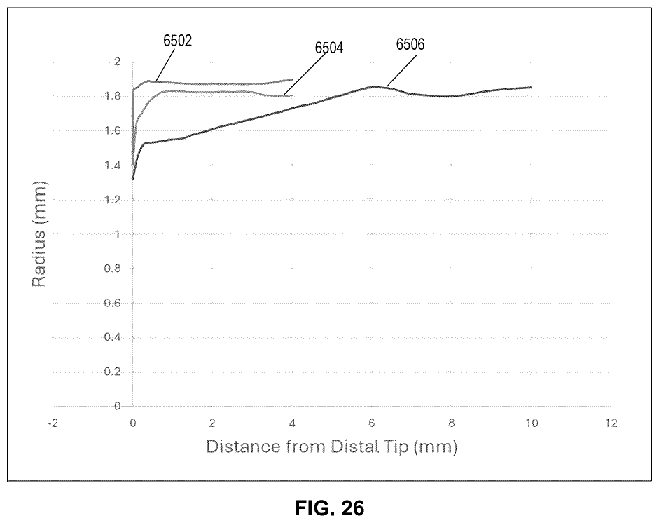

FIG. 26 is a graph comparing change in radius of different sheaths of septum puncture devices over a distance from a distal tip of the sheaths, according to embodiments. As shown, a first sheath 6502 of a first septum puncture device and a second sheath 6504 of a second septum puncture device have a sharp increase in radius (i.e., corresponding to an outer diameter of the sheaths). The sheaths 6502, 6504 reach a maximum radius within less than 1 mm from a distal tip of the sheath. Therefore, the sheaths 6502, 6504 create a step or ledge from the dilator to the sheath. In contrast, a third sheath 6506 corresponding to the sheaths described herein (e.g., with respect to FIGS. 24A-25) shows a gradual increase in radius from the distal end of the sheath. The radius of the third sheath 6506 reaches a maximum diameter at about 6 mm from a distal end of the sheath, and an average slope of the taper is about 0.08. A gradual taper having an average slope less than 0.12 reduces tissue shear forces (and therefore risk of tissue trauma) during dilation compared to abrupt step transitions. This feature can allow for a tight transition (e.g., no step) over a smaller dilator while also allowing a larger catheter such as our extendable catheter or a therapy catheter to pass though the tip. In other words, the third sheath tip 6506 allows for a smooth, atraumatic septal crossing while also enabling advancement of a catheter having an end effector feature. A large step from the dilator to the fixed sheath can result in high crossing forces, potentially buckling the sheath. Therefore, the gradual taper of the third sheath 6506 can also reduce a force level for crossing the septum, thereby preventing buckling or kinking of the sheath.

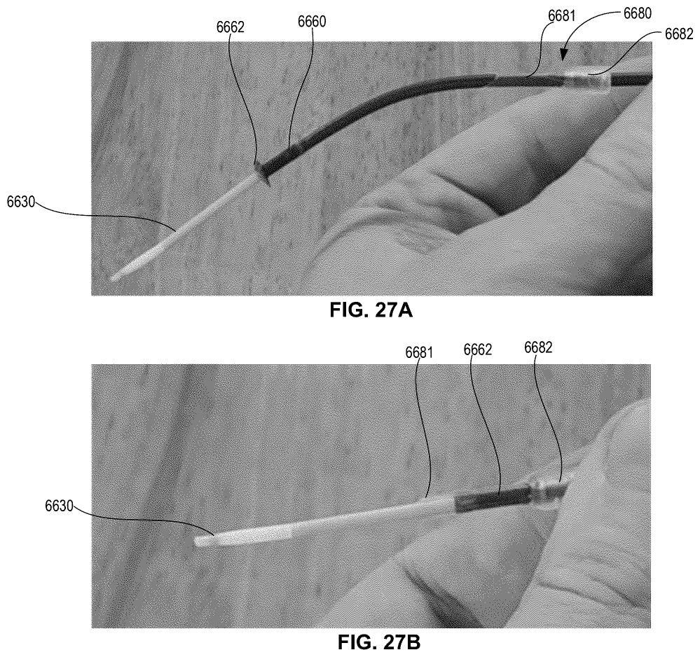

FIGS. 27A-27B show a catheter unconstrained and constrained, respectively, by a loading tool 6680, according to embodiments. FIG. 27A shows a catheter 6660 of a septum puncture device including an end effector 6662 at a distal end thereof in an expanded or unconstrained configuration. The catheter 6660 defines a lumen through which a dilator 6630 can be disposed. During loading of the catheter 6660, the dilator 6630 can be extended distal to the catheter 6660. In some embodiments, a loading tool 6680 can include a distal lumen 6681 and a hub 6682 configured to be engaged by a user. In some embodiments, the loading tool 6680 can be configured to be disposed about a proximal end of the catheter 6660 and advanced distally over the catheter 6660. The loading tool 6680 can be configured to transition the end effector 6662 from the unconstrained or expanded configuration to the constrained or unexpanded configuration. In some embodiments, the loading tool 6680, when advanced distally, can be configured to push the end effector distally to fold the end effector into a cylinder (i.e., from a larger diameter to a smaller diameter). The loading tool 6680 can constrain the end effector to a diameter smaller than an inner diameter of the sheath such that the catheter 6660 can be loaded into the sheath.

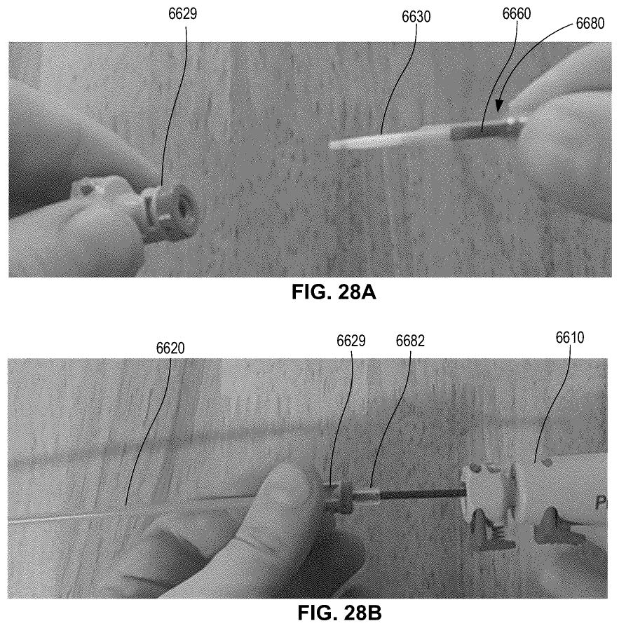

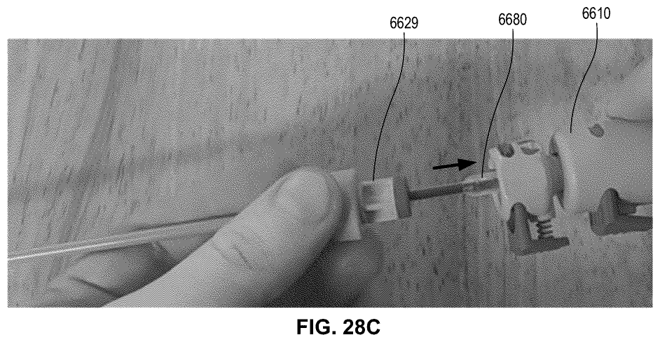

FIGS. 28A-28C show loading of the catheter of FIGS. 27A-27B into a proximal end 6629 of a sheath 6620 of a puncture septum device, according to embodiments. As shown, the catheter 6660 with the loading tool 6680 disposed around a distal end thereof is configured to be disposed through the proximal end 6629 of the sheath 6620. The catheter 6660 can be disposed through the proximal end 6629 of the sheath 6620 until the proximal end 6629 of the sheath 6620 abuts the hub 6682 of the loading tool 668, at which point, the end effector is disposed within the sheath 6620. A handle assembly 6610 can be advanced over a proximal end of the catheter 6660 to couple the handle assembly 6610 to the proximal end 6629 of the sheath 6620. In some embodiments, the handle assembly 6610 can define a volume configured to receive the loading tool 6680 during the procedure. For example, once the end effector is loaded into the sheath, the loading tool 6680 can be withdrawn proximally into a distal portion of the handle assembly 6610.

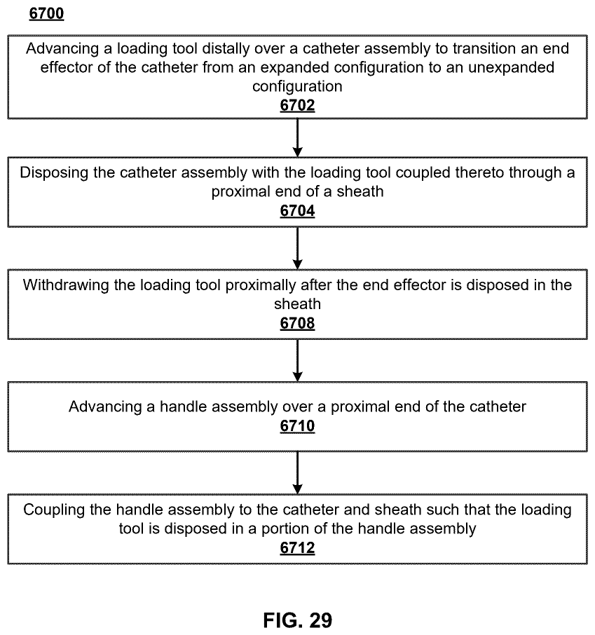

FIG. 29 is a schematic block diagram of a method 6700 of loading a catheter into a sheath, according to some embodiments. In some embodiments, the method 6700 can include advancing a loading tool distally over a catheter assembly to transition an end effector of the catheter from an expanded configuration to an unexpanded configuration, at 6702. The loading tool can be configured to transition the end effector to a diameter less than an inner diameter of a sheath. In some embodiments, the method 6700 can include folding a portion of the end effector distally such that the end effector forms a cylinder. The catheter assembly can include a catheter defining a lumen configured to slidably receive a dilator therethrough. The dilator can include a lumen configured to receive an RF guidewire therethrough. The method 6700 can include disposing the catheter assembly with the loading tool coupled thereto through a proximal end of a lumen of a sheath, at 6704. In some embodiments, the method 6700 includes withdrawing the loading tool proximally after the end effector is disposed in the sheath, at 6708. In some embodiments, the method 6700 can include advancing a handle assembly over a proximal end of the catheter, at 6710. In some embodiments, the method can include coupling the handle assembly to the proximal end of the catheter and the sheath such that the loading tool is disposed in a portion of the handle assembly, at 6712. In some embodiments, the sheath, catheter, dilator, and guidewire can be structurally and/or functionally similar to any of the sheaths, catheter, dilators, and guidewires described herein. In some embodiments, once the catheter is loaded through the sheath, a transseptal puncture procedure can be performed (e.g., as described in FIGS. 21A-21K and/or FIG. 23.

Detailed embodiments of the present disclosure have been disclosed herein or purposes of describing and illustrating claimed structures and methods that can be embodied in various forms, and are not intended to be exhaustive in any way, or limited to the disclosed embodiments. Many modifications and variations will be apparent without departing from the scope of the disclosed embodiments. The terminology used herein was chosen to best explain the principles of the one or more embodiments, practical applications, or technical improvements over current technologies, or to enable understanding of the embodiments disclosed herein. As described, details of well-known features and techniques can be omitted to avoid unnecessarily obscuring the embodiments of the present disclosure.

References in the specification to “one embodiment,” “an embodiment,” “an example embodiment,” or the like, indicate that the embodiment described can include one or more particular features, structures, or characteristics, but it shall be understood that such particular features, structures, or characteristics may or may not be common to each and every disclosed embodiment disclosed herein. Moreover, such phrases do not necessarily refer to any one particular embodiment per se. As such, when one or more particular features, structures, or characteristics is described in connection with an embodiment, it is submitted that it is within the knowledge of those skilled in the art to affect such one or more features, structures, or characteristics in connection with other embodiments, where applicable, whether or not explicitly described.

Parameters, dimensions, materials, and configurations described herein are meant to be examples and that the actual parameters, dimensions, materials, and/or configurations will depend upon the specific application or applications for which the inventive teachings is/are used. It is, therefore, to be understood that the foregoing embodiments are presented by way of example only and that, within the scope of the appended claims and equivalents thereto; and that embodiments can be practiced otherwise than as specifically described and claimed. Embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

As you herein, the phrase “and/or” should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” phrase, whether related or unrelated to those elements specifically identified. Thus, as a non-limiting example, a reference to “A and/or B”, when used in conjunction with open-ended language such as “comprising” or “including” can refer, in one embodiment, to A only (optionally including elements other than B); in another embodiment, to B only (optionally including elements other than A); in yet another embodiment, to both A and B (optionally including other elements); etc.

As used herein, the term, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein, the terms “about” and/or “approximately” when used in conjunction with values and/or ranges generally refer to those values and/or ranges near to a recited value and/or range. In some instances, the terms “about” and “approximately” may mean within +10% of the recited value. For example, in some instances, “approximately a diameter of an instrument” may mean within +10% of the diameter of the instrument. The terms “about” and “approximately” may be used interchangeably. Similarly, the term “substantially” when used in conjunction with physical and/or geometric feature(s), structure(s), characteristic(s), relationship(s), etc. is intended to convey that the feature(s), structure(s), characteristic(s), relationship(s), etc. so defined is/are nominally the feature(s), structure(s), characteristic(s), relationship(s), etc. As one example, a first quantity that is described as being “substantially equal” to a second quantity is intended to convey that, although equality may be desirable, some variance can occur. Such variance can result from manufacturing tolerances, limitations, approximations, and/or other practical considerations. Thus, the term “substantially.”

While various embodiments have been described above, it should be understood that they have been presented by way of example only, and not limitation. Where schematics and/or embodiments described above indicate certain components arranged in certain orientations or positions, the arrangement of components may be modified. While the embodiments have been particularly shown and described, it will be understood that various changes in form and details may be made. Although various embodiments have been described as having particular features and/or combinations of components, other embodiments are possible having a combination of any features and/or components from any of embodiments described herein.

The specific configurations of the various components can also be varied. For example, the size and specific shape of the various components can be different from the embodiments shown, while still providing the functions as described herein. More specifically, the size and shape of the various components can be specifically selected for a desired or intended usage. Thus, it should be understood that the size, shape, and/or arrangement of the embodiments and/or components thereof can be adapted for a given use unless the context explicitly states otherwise.

Where methods and/or events described above indicate certain events and/or procedures occurring in certain order, the ordering of certain events and/or procedures may be modified. Additionally, certain events and/or procedures may be performed concurrently in a parallel process, when possible, as well as performed sequentially as described above.

1. An apparatus, comprising:
a sheath defining a lumen;
a catheter slidably disposable within the lumen of the sheath and configured to extend distally relative to the sheath, the catheter defining a lumen;
a dilator slidably disposable within the lumen of the catheter and configured to extend distally relative to the catheter and the sheath, the dilator defining a lumen,
a distal end portion of the sheath has an inner diameter that is less than a maximum outer diameter of the dilator when a portion of the dilator having the maximum outer diameter is disposed proximal to or distal to the distal end portion of the sheath;
a radiofrequency (RF) guidewire slidably disposable within the lumen of the dilator and configured to extend distally relative to the dilator, a proximal end portion of the RF guidewire configured to be coupled to an RF generator to deliver RF energy to a distal end portion of the RF guidewire and to a target perforation site of a patient.

2. The apparatus of claim 1, wherein the catheter includes an end effector disposed at a distal end thereof, the end effector is configured to transition between a first configuration in which the end effector has a first diameter and a second configuration in which the end effector has a second diameter greater than the first diameter.

3. The apparatus of claim 2, wherein the end effector forms an atraumatic shape in the second configuration.

4. The apparatus of claim 2, wherein the end effector is configured to automatically transition from the first configuration to the second configuration as the end effector is advanced distal to the sheath.

5. The apparatus of claim 2, wherein the end effector is echogenic.

6. The apparatus of claim 2, wherein the end effector is formed of braided nitinol.

7. The apparatus of claim 1, wherein the end effector in the first configuration extends distally from the catheter, a distal end of the end effector is configured to move proximally when the end effector transitions from the first configuration to the second configuration.

8. The apparatus of claim 1, wherein the distal end portion of the sheath has a lower durometer than a portion of the sheath proximal to the distal end portion of the sheath.

9. The apparatus of claim 8, wherein the distal end portion of the sheath having the lower durometer is configured to expand laterally to accommodate the maximum outer diameter of the dilator as the dilator is advanced through the sheath.

10. The apparatus of claim 9, wherein the distal end portion of the sheath having the lower durometer is configured to form a seal with an outer surface of the dilator as a portion of the dilator is advanced distal to the sheath.

11. The apparatus of claim 1, wherein an outer diameter of the distal end portion of the sheath tapers from a first diameter to a second diameter smaller than the first diameter.

12. An apparatus, comprising:
a sheath defining a lumen;
a catheter slidably disposable within the lumen of the sheath and configured to extend distally relative to the sheath, the catheter defining a lumen and having an end effector at a distal end thereof, the end effector configured to transition from first configuration in which the end effector has a first diameter to a second, atraumatic configuration in which the end effector has a second diameter greater than the first diameter;
a dilator slidably disposable within the lumen of the catheter and configured to extend distally relative to the catheter and the sheath, the dilator defining a lumen;
a radiofrequency (RF) guidewire slidably disposable within the lumen of the dilator and configured to extend distally relative to the dilator, the distal end portion of the RF guidewire configured to be activated to deliver RF to perforate a target perforation site of a patient.

13. The apparatus of claim 12, wherein the end effector is configured to automatically transition from the first configuration to the second configuration as the end effector is advanced distal to the sheath.

14. The apparatus of claim 12, wherein the end effector is echogenic.

15. The apparatus of claim 12, wherein the end effector is formed of braided nitinol.

16. The apparatus of claim 12, wherein the dilator includes a shapeable hypotube.

17. The apparatus of claim 12, wherein the distal end portion of the sheath has a lower durometer than a portion of the sheath proximal to the distal end portion of the sheath.

18. The apparatus of claim 17, wherein the distal end portion of the sheath having the lower durometer is configured to expand laterally to accommodate a maximum outer diameter of the dilator as the dilator is advanced through the sheath.

19. The apparatus of claim 18, wherein the distal end portion of the sheath having the lower durometer is configured to form a seal with an outer surface of the dilator as a portion of the dilator is advanced distal to the sheath.

20. The apparatus of claim 12, wherein an outer diameter of the distal end portion of the sheath tapers from a first diameter to a second diameter smaller than the first diameter.

21. An apparatus, comprising:
a sheath;
a dilator slidably disposable within the sheath and configured to extend distally relative to the sheath, the dilator defining a lumen,
a distal end portion of the sheath having a lower durometer than a portion of the sheath proximal to the distal end portion of the sheath such that an inner diameter of the distal portion of the sheath is configured to expand and contract laterally according to an outer diameter of the dilator; and
a radiofrequency (RF) guidewire slidably disposable within the lumen of the dilator and configured to extend distally relative to the dilator, the distal end portion of the RF guidewire configured to be activated to deliver RF to perforate a target perforation site of a patient.

22. The apparatus of 21, further comprising:
a catheter slidably disposable within a lumen of the sheath and configured to extend distally relative to the sheath.

23. The apparatus of claim 22, wherein the dilator is slidably disposed within a lumen of the catheter and configured to extend distally relative to the catheter and the sheath.

24. The apparatus of claim 23, wherein the catheter includes an end effector at a distal end thereof, the end effector configured to transition from an unexpanded configuration to an expanded configuration when disposed distal to the sheath.

25. The apparatus of claim 24, wherein the end effector in the first configuration extends distally from the catheter, a distal end of the end effector is configured to move proximally when the end effector transitions from the first configuration to the second configuration.

26. The apparatus of claim 21, wherein the distal end portion of the sheath has an inner diameter that is less than a maximum outer diameter of the dilator when a portion of the dilator having the maximum outer diameter is disposed proximal to the distal end portion of the sheath

27. A method, comprising:
navigating a catheter assembly to a right atrium of a patient, the catheter assembly including a sheath and a catheter slidably disposed in the sheath, the catheter including an end effector on a distal end thereof;
disposing the end effector of the catheter distal to a distal end of the sheath such that the end effector transitions from a first configuration in which the end effector has a first diameter to a second, atraumatic configuration in which the end effector has a second diameter greater than the first diameter;
tenting a target perforation site of a septum with the end effector; and
perforating, with the target perforation site tented, the target perforation site with the guidewire.

28. The method of claim 27, further comprising:
dilating by advancing a dilator and the sheath along the RF guidewire through the perforation site and into a left atrium, the dilator being slidably disposed in a lumen of the catheter.

29. The method of claim 28, further comprising:
after dilating, withdrawing the dilator and the RF guidewire proximally within the sheath and from the left atrium, leaving the distal end portion of the sheath within the left atrium.

30. The method of claim 28, wherein a distal end portion of the sheath has an inner diameter that is less than an outer maximum diameter of the dilator when a portion of the dilator having the maximum outer diameter is disposed proximal to or distal to the distal end portion of the sheath.
